(19) United States
(12) Patent Application Publication
(10) Pub. No.: US 2008/0051635 A1
(43) Pub. Date: Feb. 28, 2008

(54) Capsule-type medical apparatus and drug delivery system using the same

(75) Inventors: Shinsuke Tanaka, Tokyo (JP); Hironao Kawano, Tokyo (JP); Akira Kikuchi, Yokohama-shi (JP); Hirofumi Tsuboi, Tokyo (JP); Ryoji Sato, Tokyo (JP); Hironobu Takizawa, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corp., Tokyo (JP)

(21) Appl. No.: 11/893,882

(22) Filed: Aug. 16, 2007

(30) Foreign Application Priority Data
May 23, 2006 (JP) 2006-143186

(51) Int. Cl. A61B 1/06

(52) U.S. Cl. 600/160

(57) ABSTRACT

A capsule-type medical apparatus holds a drug such that a drug release such as dissolution can be realized under the same condition as in a case where the drug is delivered to an interior of a living body by itself, and realizes confirmation on whether the drug delivered to the interior of the living body is actually released to a site in the living body or not. The capsule-type medical apparatus includes a capsule-like casing which is formed in a suitable size for insertion into the living body, a net-like drug holding unit which houses a drug in a releasable manner with respect to a site in the living body, and an imaging unit which captures an image covering the drug held in the drug holding unit and a surrounding area of the drug. The imaging unit 4 captures an image as drug-source information indicating a release condition of the drug with respect to the site in the living body.

### BACKGROUND OF THE INVENTION

1. Field of the Invention

The present invention relates to a capsule-type medical apparatus which is inserted into an interior of a living body to deliver a drug to a site in the living body, and to a drug delivery system including the same.

2. Description of the Related Art

Conventionally, some medical treatments are performed by inserting an endoscope into an interior of a living body of, for example, a patient, and by delivering a drug to a site in the living body with the use of the inserted endoscope. The drug is inserted into or injected into an internal duct (e.g. channel) of the endoscope penetrating through the interior of the living body, and delivered to a site (e.g., internal organ such as stomach or duodenum) in the living body through the internal duct of the endoscope (see Japanese Patent Application Laid-Open No. H5-297289).

In the field of endoscope, a drug delivery system is proposed in recent years for delivering a drug to an interior of a living body with the use of a capsule-type medical apparatus formed in a suitable size for insertion into the living body. The capsule-type medical apparatus for such a drug delivery system, which stores a drug inside a compartment having a plurality of holes, is swallowed by a living body from the mouth, and moves through the digestive tract of the living body while delivering the drug to a desirable site (see description of United States Patent Application Publication No. 2005/0137468). The drug delivered to the interior of the living body dissolves in a body fluid to be released to the site in the living body. Such a capsule-type medical apparatus can alleviate pains of the living body at a time of drug delivery, and realizes drug delivery to a deep portion in the living body (e.g., the small intestine), to which it is difficult to deliver a drug using such an elongated endoscope as mentioned above.

In an application of the drug delivery system using the capsule-type medical apparatus for delivering a drug to an interior of the living body, a drug to be delivered inside the living body is tested outside the living body in advance for its solubility. Based on a result of the test, an inference is made on whether the drug dissolves at a site in the living body or not (in other words, whether the drug is released to the site in the living body or not). With respect to the drug delivery system as described above, it is desirable that a check can be done on whether the drug delivered to the interior of the living body by the capsule-type medical apparatus is actually released to the site in the living body, rather than relying on a mere inference.

Further, in research and development of a drug which selectively dissolves at a predetermined site in the living body, it is similarly desirable to provide means for checking whether the drug delivered to the interior of the living body actually dissolves at a target site in the living body or not.

Still further, in the capsule-type medical apparatus described in United States Patent Application Publication No. 2005/0137468, the drug is made to contact with a body fluid through holes of the compartment housing the drug. Therefore, a contact condition of the drug and the body fluid is different from a case where the drug is delivered to the interior of the living body by itself. Hence, the state change (e.g., dissolution) of the drug in the compartment is different from the state change of the drug delivered to the interior of the living body by itself.

### SUMMARY OF THE INVENTION

An object of the present invention is at least to solve the problems as described above.

A capsule-type medical apparatus according to one aspect of the present invention has a capsule-like casing that can be inserted into an interior of a living body and delivers a drug to a site in the living body, and the capsule-type medical apparatus includes a holding unit that holds the drug in such a manner that the drug can be brought into contact with a tissue in the living body, and a detecting unit that detects change in the drug in the living body.

A drug delivery system according to another aspect of the present invention includes a capsule-type medical apparatus that is inserted into an interior of a living body, holds and releases a drug to a site in the living body, and detects drug-source information indicating at least a release condition of the drug at the site in the living body, and a display unit that displays the drug-source information detected by the capsule-type medical apparatus.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram of an exemplary configuration of a capsule-type medical apparatus according to a first embodiment of the present invention;

FIG. 2 is a schematic block diagram of an exemplary configuration of the capsule-type medical apparatus according to the first embodiment of the present invention;

FIG. 3 is a schematic diagram of an exemplary configuration of a drug delivery system including the capsule-type medical apparatus according to the first embodiment of the present invention;

FIG. 4 is a schematic diagram illustrating a state of the capsule-type medical apparatus according to the first embodiment inserted into an interior of a living body;

FIG. 5 is a schematic diagram of a specific example of an image captured by the capsule-type medical apparatus according to the first embodiment;

FIG. 6 is a schematic diagram of an exemplary configuration of a capsule-type medical apparatus according to a first modification of the first embodiment of the present invention;

FIG. 7 is a schematic diagram of one example of a folded state of a connecting member;

FIG. 8 is a schematic diagram of an exemplary configuration of a drug delivery system including the capsule-type medical apparatus according to the first modification of the first embodiment of the present invention;

FIG. 9 is a schematic diagram illustrating a state of the capsule-type medical apparatus according to the first modification of the first embodiment inserted into the interior of the living body;

FIG. 10 is a schematic diagram of a specific example of an image captured by the capsule-type medical apparatus according to the first modification of the first embodiment;

FIG. 11 is a schematic diagram of an exemplary configuration of a capsule-type medical apparatus according to a second modification of the first embodiment of the present invention;

FIG. 12 is a schematic diagram of an exemplary configuration of a drug delivery system including the capsule-type medical apparatus according to the second modification of the first embodiment of the present invention;

FIG. 13 is a schematic diagram illustrating a state of the capsule-type medical apparatus according to the second modification of the first embodiment inserted into the interior of the living body;

FIG. 14 is a schematic diagram of a specific example of an image captured by the capsule-type medical apparatus according to the second modification of the first embodiment;

FIG. 15 is a schematic diagram of an exemplary configuration of a capsule-type medical apparatus according to a third modification of the first embodiment of the present invention;

FIG. 16 is a schematic diagram of an exemplary configuration of a drug delivery system including the capsule-type medical apparatus according to the third modification of the first embodiment of the present invention;

FIG. 17 is a schematic diagram illustrating a state of the capsule-type medical apparatus according to the third modification of the first embodiment inserted into the interior of the living body;

FIG. 18 is a schematic diagram of a specific example of an image captured by the capsule-type medical apparatus according to the third modification of the first embodiment;

FIG. 19 is a schematic diagram of an exemplary configuration of a capsule-type medical apparatus according to a second embodiment of the present invention;

FIG. 20 is a schematic block diagram of an exemplary configuration of the capsule-type medical apparatus according to the second embodiment of the present invention;

FIG. 21 is a schematic diagram of an exemplary configuration of a drug delivery system including the capsule-type medical apparatus according to the second embodiment of the present invention;

FIG. 22 is a schematic diagram illustrating a state of the capsule-type medical apparatus according to the second embodiment inserted into the interior of the living body;

FIG. 23 is a schematic diagram of a specific example of an image captured by the capsule-type medical apparatus according to the second embodiment;

FIG. 24 is a schematic diagram of an exemplary configuration of a capsule-type medical apparatus according to a third embodiment of the present invention;

FIG. 25 is a schematic diagram of an example of a disassembled state of a capsule-like casing;

FIG. 26 is a schematic block diagram of an exemplary configuration of the capsule-type medical apparatus according to the third embodiment of the present invention;

FIG. 27 is a schematic diagram of an exemplary configuration of a drug delivery system including the capsule-type medical apparatus according to the third embodiment of the present invention;

FIG. 28 is a schematic diagram illustrating a state of the capsule-type medical apparatus according to the third embodiment inserted into the interior of the living body;

FIG. 29 is a schematic diagram of an exemplary configuration of a capsule-type medical apparatus according to a fourth embodiment of the present invention;

FIG. 30 is a schematic block diagram of an exemplary configuration of the capsule-type medical apparatus according to the fourth embodiment of the present invention;

FIG. 31 is a schematic diagram of an exemplary configuration of a drug delivery system including the capsule-type medical apparatus according to the fourth embodiment of the present invention;

FIG. 32 is a schematic diagram illustrating a state of the capsule-type medical apparatus according to the fourth embodiment inserted into the interior of the living body;

FIG. 33 is a schematic diagram of an exemplary configuration of a capsule-type medical apparatus according to a fifth embodiment of the present invention;

FIG. 34 is a schematic block diagram of an exemplary configuration of the capsule-type medical apparatus according to the fifth embodiment of the present invention;

FIG. 35 is a schematic diagram of an exemplary configuration of a drug delivery system including the capsule-type medical apparatus according to the fifth embodiment of the present invention;

FIG. 36 is a schematic diagram illustrating a state of the capsule-type medical apparatus according to the fifth embodiment collecting a body fluid inside the living body;

FIG. 37 is a schematic diagram of an exemplary configuration of a capsule-type medical apparatus according to a sixth embodiment of the present invention;

FIG. 38 is a schematic block diagram of an exemplary configuration of the capsule-type medical apparatus according to the sixth embodiment of the present invention;

FIG. 39 is a schematic diagram of an exemplary configuration of a drug delivery system including the capsule-type medical apparatus according to the sixth embodiment of the present invention;

FIG. 40 is a schematic diagram illustrating a state of the capsule-type medical apparatus according to the sixth embodiment collecting the body fluid at one site in the living body plural times;

FIG. 41 is a schematic diagram of an exemplary configuration of a capsule-type medical apparatus according to a seventh embodiment of the present invention;

FIG. 42 is a schematic block diagram of an exemplary configuration of the capsule-type medical apparatus according to the seventh embodiment of the present invention;

FIG. 43 is a schematic diagram of an exemplary configuration of a drug delivery system including the capsule-type medical apparatus according to the seventh embodiment of the present invention;

FIG. 44 is a schematic diagram illustrating a state of the capsule-type medical apparatus according to the seventh embodiment collecting the body fluid at each site in the living body;

FIG. 45 is a schematic diagram of an example of a capsule-type medical apparatus in which a capsule-like casing and a drug can be detachably connected with each other via a connecting member; and

FIG. 46 is a schematic diagram illustrating a connected state of a drug having a hole and a drug holding unit.

### DETAILED DESCRIPTION OF THE PREFERRED EMBODIMENTS

Exemplary embodiments of a capsule-type medical apparatus and a drug delivery system including the same according to the present invention will be described in detail below with reference to the accompanying drawings. It should be noted that the present invention is not limited by the following embodiments.

### First Embodiment

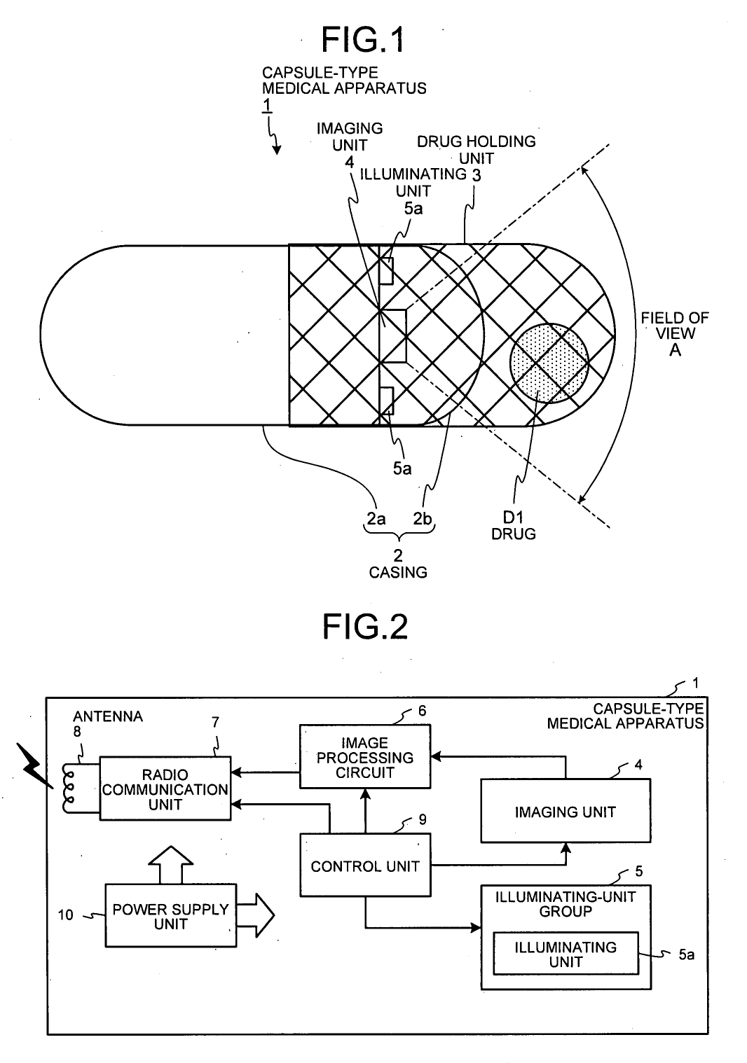

FIG. 1 is a schematic diagram of an exemplary configuration of a capsule-type medical apparatus according to a first embodiment of the present invention. FIG. 2 is a schematic block diagram of the exemplary configuration of the capsule-type medical apparatus according to the first embodiment. As shown in FIGS. 1 and 2, a capsule-type medical apparatus 1 according to the first embodiment includes a casing 2 that is formed in a capsule-like shape, a drug holding unit 3 that holds a drug D1 to be delivered to an interior of a living body, an imaging unit 4 that captures images of the drug D1 held by the drug holding unit 3, and plural illuminating units 5a that illuminate a field of view A of the imaging unit 4. Further, the capsule-type medical apparatus 1 includes an image processing circuit 6 that generates image signals including images captured by the imaging unit 4, a radio communication unit 7 and an antenna 8 that serve for radio communication of the images captured by the imaging unit 4, a control unit 9 that controls driving of each component of the capsule-type medical apparatus 1, and a power supply unit 10 that supplies driving power to each component of the capsule-type medical apparatus 1.

The casing 2 is a capsule-like casing which is formed in a suitable size for the insertion into the living body. Specifically, the casing 2 includes a casing main body 2a which is formed in a capsule-like shape, and an optical dome 2b which is attached to a front end of the casing main body 2a. The casing main body 2a is a cylindrical casing whose front end side is open and whose back end side is closed in a dome-like shape. The casing main body 2a houses each component of the capsule-type medical apparatus 1 inside. The optical dome 2b is a substantially transparent dome-like member having a high optical transparency. The optical dome 2b is attached to the front end of the casing main body 2a and thereby closes the open end of the casing main body 2a. A living body, such as a patient, can easily swallow the casing 2 formed from the casing main body 2a and the optical dome 2b from the mouth, and the casing 2 can easily move inside the digestive tract of the living body following the peristaltic movements, for example.

The drug holding unit 3 serves as a holding unit that holds the drug D1 in a releasable manner with respect to a site inside the living body. Specifically, the drug holding unit 3 is a net-like member having plural meshes, for example, and is attached to the casing 2 so as to enclose the drug D1 and to cover the optical dome 2b. The drug holding unit 3 is formed in a form of a bag or a basket having meshes and is attached to the casing 2 so as to close the open end thereof. The drug holding unit 3 holds the drug D1 at a position within the field of view A of the imaging unit 4 without blocking the contact between the drug D1 and the body fluid inside the living body, and transmits reflected light from the site in the living body around the drug D1 through the plural meshes to the imaging unit 4. The drug D1 held in such a manner dissolves in the body fluid flowing into the drug holding unit 3 through the plural meshes in the living body. Thus, the drug D1 dissolving in the body fluid in the living body produces a drug solution. The drug solution is released to the site in the living body through the plural meshes of the drug holding unit 3. Thus, the drug D1 held by the drug holding unit 3 is released to the site in the living body. The drug D1 is a solid drug such as a tablet and is soluble in the body fluid in the living body.

The imaging unit 4 serves as an imaging unit that captures images covering the drug D1 which decreases as being released to the site in the living body as the drug solution. Specifically, the imaging unit 4 includes a solid-state image sensor such as a charge-coupled device (CCD) or a complementary metal-oxide semiconductor (CMOS), and an optical system which focuses a subject image on a light-receiving surface of the solid-state image sensor. The imaging unit 4 has the field of view A which covers a region surrounded by the drug holding unit 3 (i.e., a position of the drug D1 held in the drug holding unit 3). The imaging unit 4 captures an image of a subject within the field of view A through the optical dome 2b every time a predetermined time elapses, for example. The imaging unit 4 sequentially captures an image covering the drug D1 which is released to the site in the living body and decreases while held by the drug holding unit 3, and the site in the living body around the drug D1 and visible through the plural meshes of the drug holding unit 3 (i.e., the site in the living body to which the drug D1 is released). The imaging unit 4 receives reflected light from the drug D1 and reflected light coming from outside the drug holding unit 3 passing through the meshes towards the imaging unit 4 within the field of view A (i.e., reflected light from a surrounding area of the drug D1).

An image captured by the imaging unit 4 shows a release condition of the drug D1 which is released to the site in the living body and decreases, and the site in the living body where the drug D1 is released. Therefore, a doctor or a nurse, for example, can determine whether the drug D1 is actually released to the site in the living body or not and distinguish the site (e.g., internal organ such as stomach, duodenum, small intestine, and large intestine) where the drug D1 is released in the living body by visually confirming the image. The imaging unit 4 captures an image covering the drug D1 and the site around the drug D1 in the living body as drug-source information indicating the release condition of the drug D1 to the site in the living body and the site in the living body where the drug D1 is released. In other words, the imaging unit 4 detects the drug-source information by capturing the image covering the drug D1 and the site around the drug D1 in the living body.

An illuminating-unit group 5 includes plural illuminating units 5a for illuminating the field of view A of the imaging unit 4. The illuminating unit 5a includes, for example, a light-emitting element such as a light-emitting diode (LED), and emits illumination light to illuminate the field of view A through the optical dome 2b. Specifically, each illuminating unit 5a illuminates the drug D1 and the site around the drug D1 in the living body present within the field of view A.

The image processing circuit 6 generates image signals including the images captured by the imaging unit 4. Specifically, the image processing circuit 6 receives image data as an input from the imaging unit 4, performs predetermined image processing and the like on the received image data, and generates image signals including the image captured by the imaging unit 4 and various image parameters such as white balance. The image processing circuit 6 transmits the generated image signals to the radio communication unit 7.

The radio communication unit 7 and the antenna 8 serve as a radio transmitting unit that radio transmits the image captured by the imaging unit 4 to the outside as the drug-source information. Specifically, the radio communication unit 7 performs predetermined modulation processing and the like on the image signals supplied from the image processing circuit 6, so as to generate radio signals including the image signals. The radio communication unit 7 outputs the generated radio signals to the antenna 8. The antenna 8 is, for example, a loop antenna or a coil antenna, and transmits the radio signals supplied from the radio communication unit 7 to the outside. Thus, the radio communication unit 7 and the antenna 8 radio transmit the image captured by the imaging unit 4 to the outside.

The control unit 9 serves to control each component of the capsule-type medical apparatus 1. Specifically, the control unit 9 controls driving of each of the imaging unit 4, the illuminating-unit group 5, the image processing circuit 6, and the radio communication unit 7, and also controls input/output of various signals among respective components. For example, the control unit 9 controls the imaging unit 4 and the illuminating-unit group 5 so that timing of light emission by the plural illuminating units 5a and timing of image capture by the imaging unit 4 are synchronized. Further, the control unit 9 stores various image parameters (such as white balance) related to the images captured by the imaging unit 4.

The power supply unit 10 supplies driving power to the imaging unit 4, the illuminating-unit group 5, the image processing circuit 6, the radio communication unit 7, and the control unit 9. Further, the power supply unit 10 has a reed switch for performing an ON/OFF switching operation according to a magnetic force applied from outside, for example. The power supply unit 10 switches from an operation to start driving-power supply to an operation to stop driving-power supply and vice versa for each component of the capsule-type medical apparatus 1 according to the ON/OFF switching operation of the reed switch.

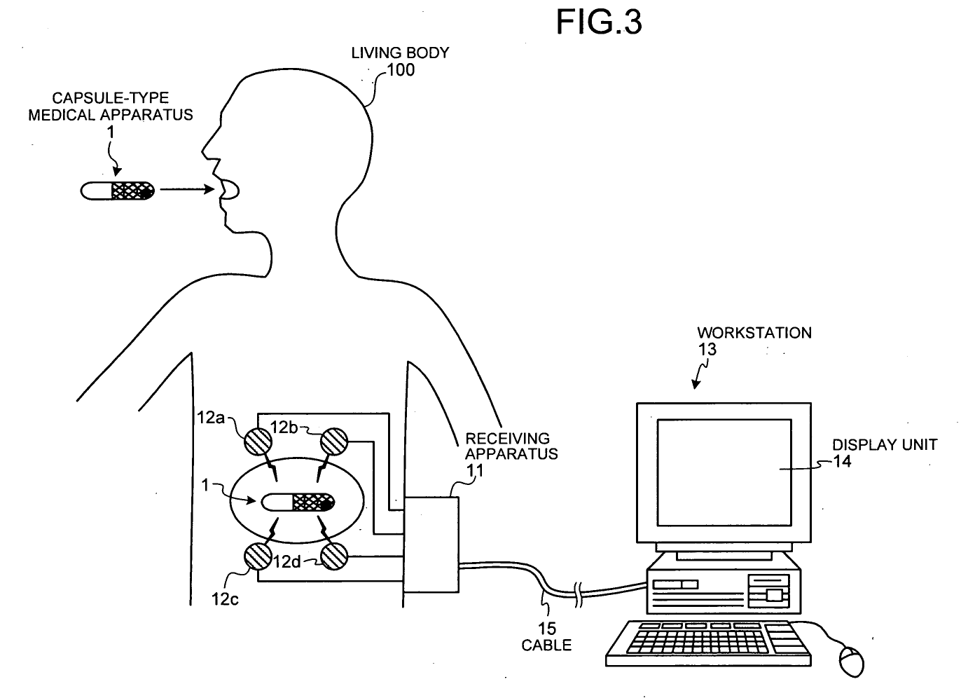

A drug delivery system including the capsule-type medical apparatus 1 according to the first embodiment of the present invention will be described below. FIG. 3 is a schematic diagram of an exemplary configuration of a drug delivery system including the capsule-type medical apparatus 1 according to the first embodiment of the present invention. As shown in FIG. 3, the drug delivery system according to the first embodiment of the present invention includes the capsule-type medical apparatus 1 which is inserted into an interior of a living body 100 together with the drug D1, a receiving apparatus 11 which receives the drug-source information radio transmitted by the capsule-type medical apparatus 1 in the living body 100, and a workstation 13 which displays the drug-source information received by the receiving apparatus 11.

The receiving apparatus 11 serves to receive the drug-source information radio transmitted by the capsule-type medical apparatus 1 inserted into the interior of the living body 100. Specifically, the receiving apparatus 11 is connected to plural receiving antennas 12a to 12d distributively arranged on a body surface of the living body 100, for example, and carried by the living body 100. The receiving apparatus 11 sequentially receives the radio signals transmitted from the capsule-type medical apparatus 1 in the living body 100 via any one of the plural receiving antennas 12a to 12d, and sequentially acquires the drug-source information (i.e., images covering the drug D1 and the site around the drug D1 in the living body) based on the received radio signals.

The receiving antennas 12a to 12d are, for example, loop antennas. The receiving antennas 12a to 12d sequentially receive the radio signals transmitted from the capsule-type medical apparatus 1, and sequentially transmit the received radio signals to the receiving apparatus 11. The receiving antennas 12a to 12d are distributively arranged at predetermined positions on the body surface of the living body 100, e.g., at positions corresponding to a passage of the capsule-type medical apparatus 1 (i.e., digestive tract) within the living body 100 as shown in FIG. 3. As far as one or more receiving antennas are arranged for each living body, the number of receiving antennas is not limited to four.

The workstation 13 serves to confirm whether the drug D1 inserted into the interior of the living body 100 together with the capsule-type medical apparatus 1 is actually released to a desirable site in the living body 100 (i.e., target site of the release of the drug D1) or not. Specifically, the workstation 13 is communicatively connected to the receiving apparatus 11 via a cable 15, for example, sequentially takes in the drug-source information received by the receiving apparatus 11, and sequentially displays the obtained drug-source information, i.e., the images covering the drug D1 and the site around the drug D1 in the living body on a display unit 14. The workstation 13 displays the images (drug-source information) captured by the capsule-type medical apparatus 1 in the living body 100 on the display unit 14 in real time.

The doctor or the nurse, for example, can confirm in real time the release condition of the drug D1 at the site within the living body 100 (in other words, a decreased state of the drug D1 which is actually released to the site within the living body 100 and decreases) and the site (e.g., stomach, duodenum, small intestine, or large intestine) within the living body 100 to which the drug D1 is released by visually confirming a series of images sequentially displayed on the display unit 14 as the drug-source information. As a result, the doctor or the nurse can confirm in real time whether the drug D1 which is inserted into the interior of the living body 100 together with the capsule-type medical apparatus 1 is actually released to a desirable site within the living body 100 or not.

The workstation 13 may be connected to the receiving apparatus 11 via the cable 15 only when the images captured by the capsule-type medical apparatus 1 (i.e., drug-source information) are displayed on the display unit 14 in real time, and the cable 15 may be disconnected from the workstation 13 at other times. Thus, the living body 100 can freely move except when the drug-source information is displayed on the display unit 14 in real time. Alternatively, the workstation 13 may be communicatively connected to the receiving apparatus 11 via wireless LAN, for example. In this case, a wireless LAN communication unit, such as a wireless LAN card, may be provided in each of the receiving apparatus 11 and the workstation 13.

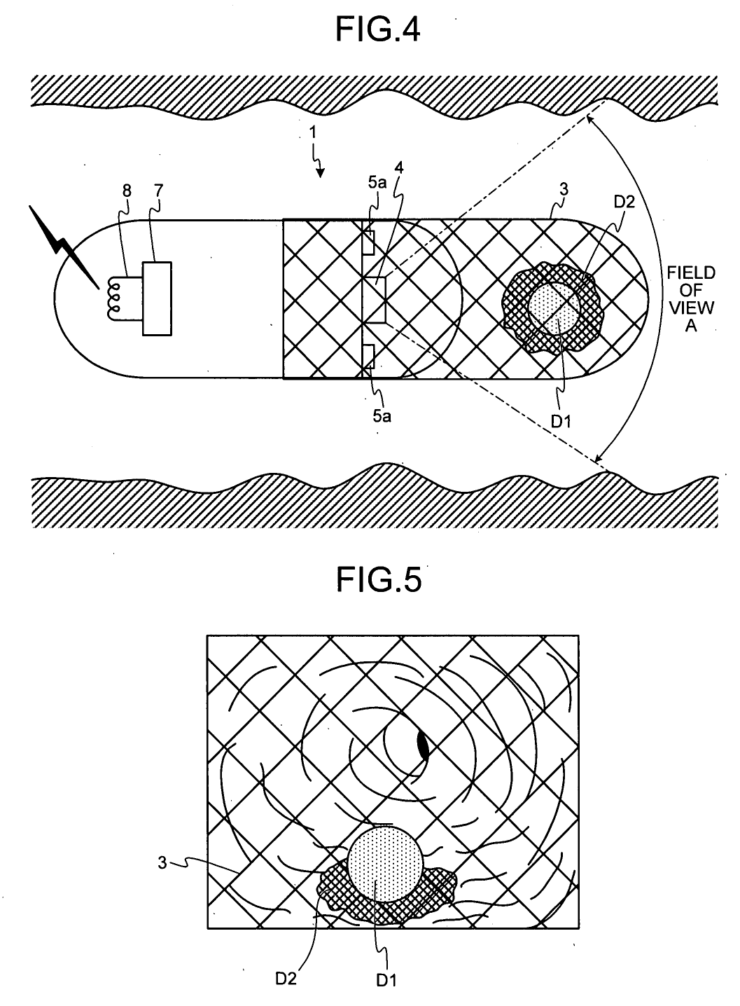

An operation of the capsule-type medical apparatus 1 inserted into the interior of the living body 100 will be described. FIG. 4 is a schematic diagram of the capsule-type medical apparatus 1 according to the first embodiment inserted into the interior of the living body. FIG. 5 is a schematic diagram of a specific example of an image captured by the capsule-type medical apparatus 1 according to the first embodiment. The operation of the capsule-type medical apparatus 1 will be described below with reference to FIGS. 4 and 5.

The capsule-type medical apparatus 1 in which the drug holding unit 3 holds the drug D1 is swallowed by the living body 100 from the mouth, for example, and thereby inserted into the interior of the living body 100 together with the drug D1. Thereafter, the capsule-type medical apparatus 1 moves through the sites in the living body 100 successively or intermittently following peristaltic movements and the like while sequentially captures images as drug-source information at predetermined intervals. The images as the drug-source information are sequentially radio transmitted to the receiving apparatus 11 outside.

Specifically, as shown in FIG. 4, the capsule-type medical apparatus 1 inserted into the interior of the living body 100 produces the drug solution D2 by bringing the drug D1 held in the drug holding unit 3 into contact with the body fluid of the living body 100 flowing into the drug holding unit 3 through meshes of the drug holding unit 3. The drug D1 in the drug holding unit 3 gradually dissolves in the body fluid and is gradually consumed for the production of the drug solution D2. The drug solution D2 is released to the site in the living body 100 through the meshes of the drug holding unit 3. Thus, the drug D1 in the drug holding unit 3 is gradually decreased while being released to the site in the living body 100 as the drug solution D2.

The imaging unit 4 always covers the drug D1 held in the drug holding unit 3 and the surrounding area of the drug D1 which is visible through the drug holding unit 3 (i.e., the site where the drug D1 is released in the living body 100) within the field of view A. Therefore, the imaging unit 4 can capture the image covering the drug D1 and the surrounding area of the drug D1. The images captured by the imaging unit 4 show, as shown in FIG. 5 for example, the drug D1 which gradually decreases as being released to the site in the living body 100 as the drug solution D2, and the surrounding area of the drug D1 visible through the drug holding unit 3. In other words, the imaging unit 4 captures an image as the drug-source information which indicates the release condition of the drug D1 to the site in the living body 100 and the site where the drug D1 is released in the living body 100.

During a period after the capsule-type medical apparatus 1 is inserted into the interior of the living body 100 until being naturally excreted from the living body 100, the imaging unit 4 sequentially captures the images as described above as the drug-source information every time a predetermined time elapses. A series of images captured as the drug-source information by the imaging unit 4 are sequentially radio transmitted via the antenna 8 by the radio communication unit 7. The series of images as the drug-source information radio transmitted from the capsule-type medical apparatus 1 are sequentially received by the receiving apparatus 11 as described above, and at the same time, sequentially taken into the workstation 13 via the cable 15, for example. Thereafter, the series of images as the drug-source information are displayed on the display unit 14 of the workstation 13 in real time.

The series of images as the drug-source information displayed on the display unit 14 in real time indicate the drug D1 which is released to the site in the living body 100 and decreased and the site within the living body 100 around the drug D1 as shown in FIG. 5. Therefore, the doctor or the nurse can confirm the decreased state of the drug D1 released to the site in the living body 100 and the site in the living body 100 to which the drug D1 is actually released (e.g., stomach, duodenum, small intestine, and large intestine) in real time by visually confirming the series of images as the drug-source information. As a result, the doctor or the nurse can confirm the release condition of the drug D1 to the site in the living body 100 in real time and check in real time whether the drug D1 is actually released to a desirable site (i.e., site such as an affected site as a target of release of the drug D1) within the living body 100 even while the capsule-type medical apparatus 1 is in the living body 100.

As described above, according to the first embodiment of the present invention, the capsule-type medical apparatus is configured to hold the drug in a releasable manner with respect to the site in the living body, to capture images covering the drug which decreases as being released to the site in the living body and a surrounding area of the drug (i.e., a site within the living body where the drug is released), and to radio transmit the image covering the drug and its surrounding area to the receiving apparatus outside the living body. Further, the capsule-type medical apparatus is configured to sequentially display the images received by the receiving apparatus on the display unit. Therefore, even while the drug is within the living body, it is possible to confirm the release condition of the drug with respect to the interior of the living body and the surrounding area of the drug in real time through the visual confirmation of the images displayed on the display unit. As a result, it is possible to provide a capsule-type medical apparatus and a drug delivery system including the capsule-type medical apparatus which realize real-time confirmation of the actual release of the drug with respect to the site in the living body and real-time confirmation of the site within the living body to which the drug is actually released.

A net-like drug holding unit (e.g., the drug holding unit 3) holding the drug in a releasable manner with respect to the site within the living body may be formed with a body-fluid-soluble material such as gelatin. When the drug holding unit is formed from a body-fluid-soluble material, the drug holding unit itself can dissolve in the living body after the release (dissolution) of the drug to the site within the living body. As a result, the capsule-type medical apparatus inside the living body can easily move through the site within the living body after releasing the drug.

Further, the net-like drug holding unit may be coated with a water-soluble material such as sugar. Then, the insertion of the capsule-type medical apparatus having the net-like drug holding unit into the living body can be further facilitated, whereby the pains of the living body can be alleviated.

### First Modification of First Embodiment

A first modification of the first embodiment of the present invention will be described. In the first embodiment described above, the drug D1 is held inside the drug holding unit 3 formed in a bag or basket shape having meshes. In the first modification of the first embodiment, the drug D1 is held by a drug case housing the drug D1 and a capsule-like casing 2 connected with each other.

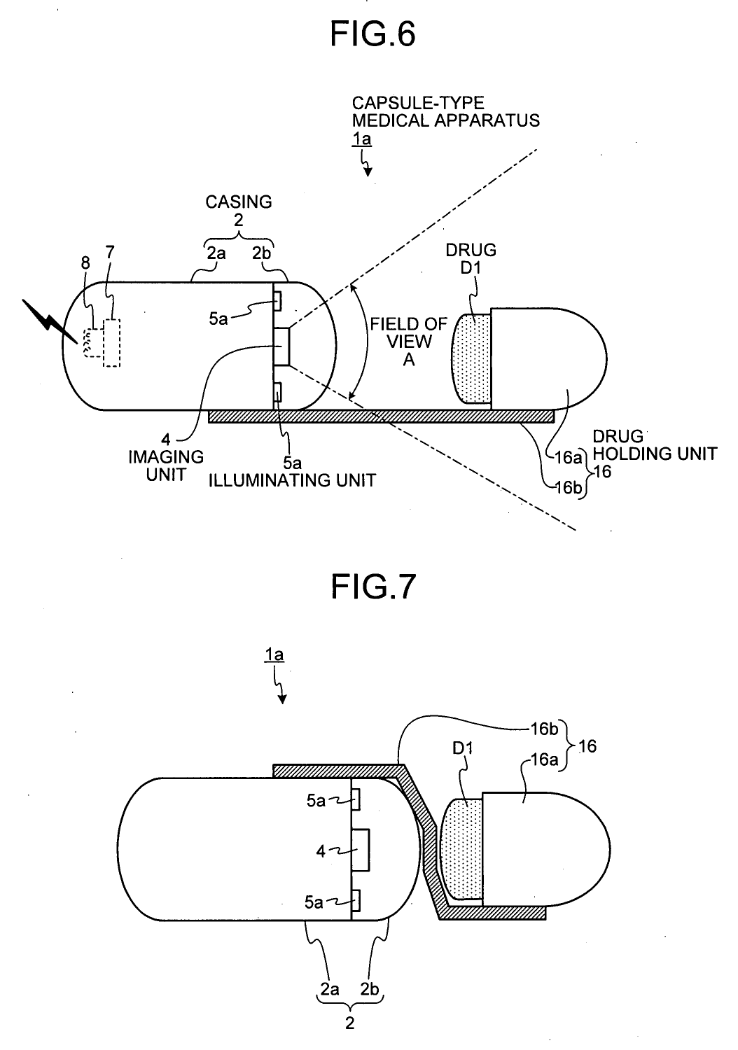

FIG. 6 is a schematic diagram of an exemplary configuration of the capsule-type medical apparatus according to the first modification of the first embodiment of the present invention. As shown in FIG. 6, a capsule-type medical apparatus la according to the first modification of the first embodiment has a drug holding unit 16 in place of the drug holding unit 3 of the capsule-type medical apparatus 1 according to the first embodiment. In other respects, the configuration of the capsule-type medical apparatus of the first modification is the same as that of the first embodiment, and the same components are denoted by the same reference characters.

The drug holding unit 16 serves as a holding unit that holds the drug D1 to be delivered to the interior of the living body in a releasable manner with respect to the site in the living body. Specifically, the drug holding unit 16 includes a drug case 16a which houses the drug D1 in a releasable manner with respect to the site in the living body and a connecting member 16b which connects the casing 2 and the drug case 16a.

The drug case 16a houses (holds) the drug D1 in an exposed state with respect to the site within the living body. In other words, the drug case 16a holds the drug D1 without blocking the contact between the drug D1 and the body fluid in the living body. The drug D1 held in the drug case 16a dissolves in the body fluid in the living body and gradually released to the site in the living body as the drug solution D2 while the amount thereof decreases.

The connecting member 16b is formed of shape memory alloy which has a predetermined shape memory characteristic and a predetermined electric resistance value. The connecting member 16b has one end connected to the casing 2 and another end connected to the drug case 16a. In other words, the connecting member 16b connects the drug case 16a housing the drug D1 and the casing 2. Thus, the connecting member 16b connects the drug D1 and the casing 2 via the drug case 16a. Further, the connecting member 16b can be deformed while maintaining the connected state of the drug case 16a and the casing 2. Specifically, the connecting member 16b can be bent or twisted while maintaining the connected state. The connecting member 16b can be folded while maintaining the connected state as shown in FIG. 7, for example. Thus, the casing 2 and the drug case 16a can be brought into contact at close proximity, and the space occupied by the capsule-type medical apparatus 1a holding the drug D1 can be made as small as possible.

Further, the connecting member 16b can be transformed into a linear shape (i.e., previously memorized shape) under a predetermined temperature condition so as to arrange the drug D1 at a position within the field of view A of the imaging unit 4. Specifically, the connecting member 16b takes an optional shape (e.g., folded state shown in FIG. 7) under the temperature condition of the temperature equal to that within the living body, for example. On the other hand, with the application of high-frequency magnetic field, inductive current is generated in the connecting member 16b. When the connecting member 16b generates heat over a predetermined temperature (i.e., temperature sufficiently higher than the temperature within the living body) due to such inductive current, the connecting member 16b changes to a linear shape and serves to arrange the drug case 16a at a predetermined position within the field of view A. The connecting member 16b arranges the drug case 16a at a predetermined position within the field of view A so as to provide an appropriate distance between the drug D1 and the imaging unit 4 for the imaging of the drug D1 by the imaging unit 4. Thus, the connecting member 16b arranges the drug D1 at a position suitable for the imaging within the field of view A by the imaging unit 4 under the predetermined temperature condition. The imaging unit 4 captures an image (i.e., image as the drug-source information) covering the drug D1 arranged at an appropriate position within the field of view A according to the function of the connecting member 16b and the site around the drug D1 in the living body.

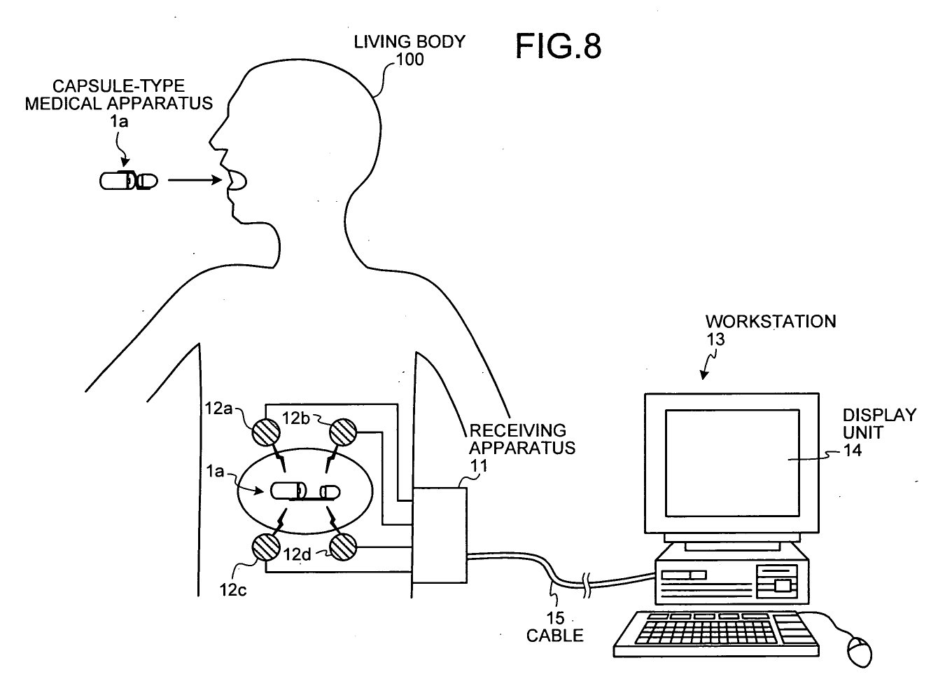

A drug delivery system including the capsule-type medical apparatus la according to the first modification of the first embodiment of the present invention will be described. FIG. 8 is a schematic diagram of an exemplary configuration of the drug delivery system including the capsule-type medical apparatus la according to the first modification of the first embodiment of the present invention. As shown in FIG. 8, the drug delivery system according to the first modification of the first embodiment of the present invention includes the capsule-type medical apparatus 1a in place of the capsule-type medical apparatus 1 of the drug delivery system according to the first embodiment. In other respects, the configuration of the drug delivery system of the first modification is the same as that of the first embodiment, and the same components are denoted by the same reference characters.

In the drug delivery system according to the first modification of the first embodiment, the capsule-type medical apparatus 1a holding the drug D1 is swallowed by the living body 100 from the mouth in a folded state as shown in FIG. 7, for example, and inserted into the living body 100 together with the drug D1. Thereafter, the capsule-type medical apparatus 1a moves through the sites in the living body 100 successively or intermittently following the peristaltic movements while sequentially capturing images as the drug-source information at predetermined intervals. The images as the drug-source information are sequentially radio transmitted to the receiving apparatus 11 outside.

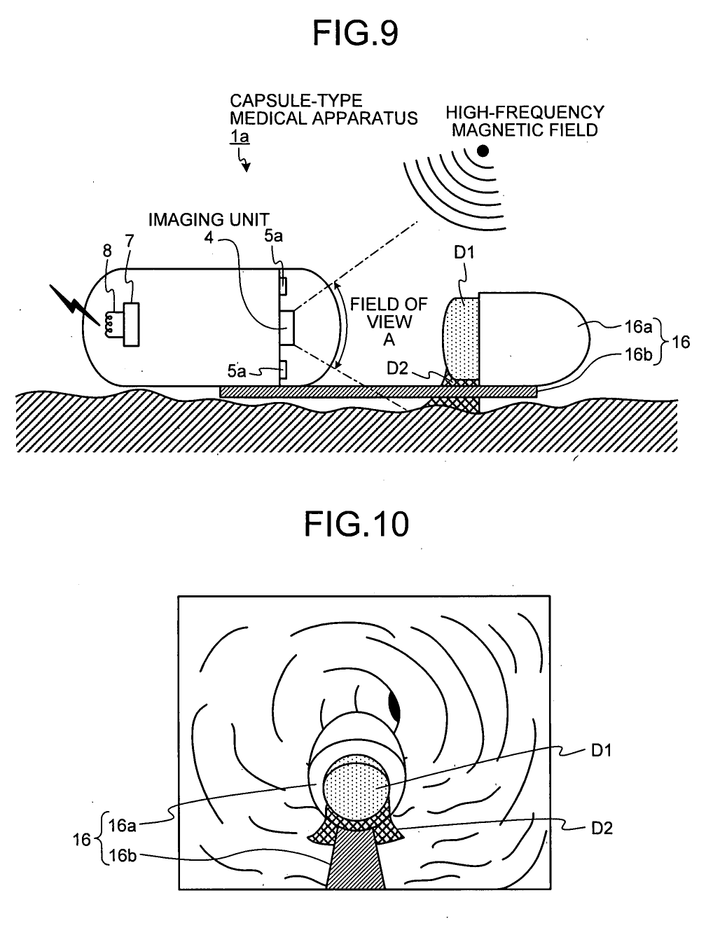

An operation of the capsule-type medical apparatus 1a in the living body 100 will be described. FIG. 9 is a schematic diagram illustrating a state of the capsule-type medical apparatus 1a according to the first modification of the first embodiment inserted into the living body. FIG. 10 is a schematic diagram of a specific example of an image captured by the capsule-type medical apparatus 1a according to the first modification of the first embodiment.

As shown in FIG. 9, the capsule-type medical apparatus 1a in the living body 100 receives an application of high-frequency magnetic field from outside the living body 100 on reaching a desirable site (site as a release target of the drug D1) within the living body 100. Due to the high-frequency magnetic field, the drug D1 is arranged at a suitable position for the imaging within the field of view A of the imaging unit 4. The connecting member 16b generates heat over a predetermined temperature due to the high-frequency magnetic field and is deformed into a linear shape so as to arrange the drug case 16a at a predetermined position within the field of view A thereby providing a suitable distance between the drug D1 and the imaging unit 4 for the imaging of the drug D1 by the imaging unit 4.

The drug D1 arranged at a suitable position for the imaging within the field of view A gradually dissolves in the body fluid in the living body 100, and is released to the site in the living body 100 as the drug solution D2, and gradually decreases. The imaging unit 4 sequentially captures images covering the drug D1 gradually decreasing and the site around the drug D1 in the living body (image as the drug-source information) at predetermined intervals.

The image captured by the imaging unit 4 shows the drug D1 which is released to the site in the living body 100 as the drug solution D2 and gradually decreases, and the site in the living body 100 visible around the drug holding unit 16 holding the drug D1 (i.e., a surrounding area of the drug D1). In other words, the imaging unit 4 captures the image as the drug-source information which indicates the release condition of the drug D1 with respect to the site in the living body 100 and the site in the living body 100 to which the drug D1 is released.

A series of images captured by the imaging unit 4 as the drug-source information are sequentially radio transmitted through the antenna 8 by the radio communication unit 7 similarly to the first embodiment, taken into the workstation 13 via the receiving apparatus 11 and the like, and displayed in real time on the display unit 14 of the workstation 13.

The series of images as the drug-source information displayed in real time on the display unit 14 show the drug D1 which is released to the site in the living body 100 and decreases and the site in the living body 100 around the drug D1 as shown in FIG. 10, for example. Therefore, the doctor or the nurse can confirm the release condition of the drug D1 with respect to the site in the living body 100 in real time, and also confirm in real time whether the drug D1 is actually released to a desirable site (i.e., a site such as an affected site as the release target of the drug D1) in the living body 100 by visually confirming the series of image as the drug-source information, similarly to the first embodiment.

As described above, in the first modification of the first embodiment, the capsule-type medical apparatus is configured so that the drug case holding the drug in a releasable manner with respect to the site in the living body and the capsule-like casing are connected with each other by the connecting member of shape memory alloy, the connecting member is deformed into a linear shape (i.e., a previously memorized shape) at a desirable site in the living body so as to arrange the drug at a position suitable for the imaging within the field of view of the imaging unit, an image covering the drug released to the site in the living body and decreases and the site around the drug is captured, and the image covering the drug and the surrounding area is radio transmitted to the receiving apparatus outside the living body. Similarly to the first embodiment, the images received by the receiving apparatus are sequentially displayed on the display unit. Therefore, in addition to the advantages and effects of the first embodiment, the first modification has an advantage that the image covering the drug released to the site in the living body and decreases and the surrounding area can be capture clearly. As a result, while the advantages and the effects of the first embodiment are similarly obtained, it is possible to provide a capsule-type medical apparatus and a drug delivery system including the capsule-type medical apparatus which realize easy confirmation of the release condition of the drug with respect to the site in the living body.

Further, since the connecting member can be deformed into a desirable shape while the connected state between the drug case and the capsule-like casing is maintained, the drug case and the capsule-like casing can be brought into close proximity with each other. As a result, a space occupied by the capsule-type medical apparatus according to the first modification of the first embodiment can be made as small as possible, whereby the easy insertion of the capsule-type medical apparatus into the living body can be realized.

### Second Modification of First Embodiment

A second modification of the first embodiment of the present invention will be described. In the first modification of the first embodiment, the drug D1 is held by the drug case 16a housing the drug D1 and the capsule-like casing 2 connected with each other via the connecting member 16b. In the second modification of the first embodiment, the drug D1 is connected to the capsule-like casing 2 via a thread-like member, whereby the drug D1 is held.

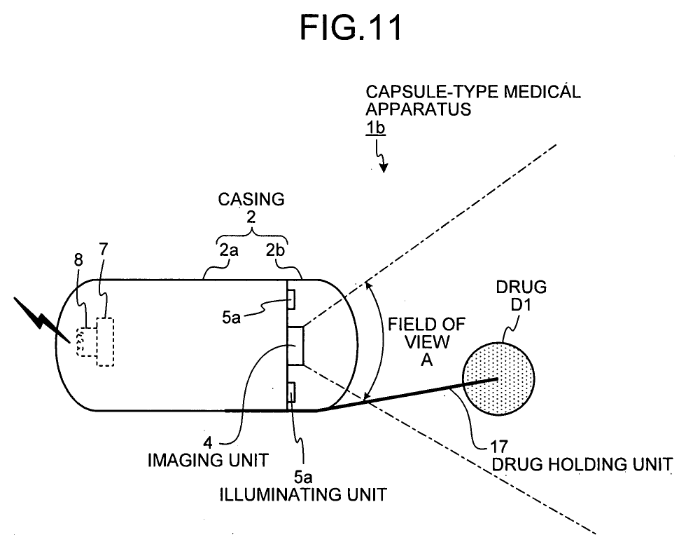

FIG. 11 is a schematic diagram of an exemplary configuration of a capsule-type medical apparatus according to the second modification of the first embodiment of the present invention. As shown in FIG. 11, a capsule-type medical apparatus 1b according to the second modification of the first embodiment includes a thread-like drug holding unit 17 in place of the drug holding unit 16 of the capsule-type medical apparatus 1a according to the first modification of the first embodiment. In other respects, the configuration of the capsule-type medical apparatus according to the second modification is the same as that of the first modification of the first embodiment, and the same components are denoted by the same reference characters.

The drug holding unit 17 serves as a holding unit that holds the drug D1 in a releasable manner with respect to the site in the living body, and also serves as a connecting unit that connects the drug D1 and the capsule-like casing 2. Specifically, the drug holding unit 17 is realized with a thread-like member whose one end is connected to the casing 2, and another end is connected to the drug D1. The drug holding unit 17 holds the drug D1 without blocking the contact between the drug D1 and the body fluid in the living body. The drug holding unit 17 brings the drug D1 into contact with the body fluid in the living body substantially similarly to the case where the drug D1 is delivered to the interior of the living body by itself. Further, the drug holding unit 17 freely deforms while maintaining the connected state of the drug D1 and the casing 2. Therefore, the drug holding unit 17 can hold the drug D1 while making a space occupied by the capsule-type medical apparatus 1b as small as possible. As a result, the pains of the living body at the time of insertion of the capsule-type medical apparatus 1b into the living body together with the drug D1 can be alleviated.

The drug D1 held by the drug holding unit 17 is arranged at a position within the field of view A of the imaging unit 4 by the drug holding unit 17 when delivered inside the digestive tract of the living body. Here, it is desirable that the capsule-type medical apparatus 1b holding the drug D1 be inserted into the interior of the living body with the casing 2 arranged at an advance direction (in other words, so that the drug D1 comes after the casing 2). Then, the drug holding unit 17 can arrange the subsequent drug D1 at a suitable position for the imaging in the field of view A. Further, the drug D1 held by the drug holding unit 17 is brought into contact with the body fluid in the living body substantially similarly to the case where the drug D1 is delivered to the interior of the living body by itself within the field of view A of the imaging unit 4. The drug D1 dissolves in the body fluid and is gradually released to the site in the living body as the drug solution D2 and decreases.

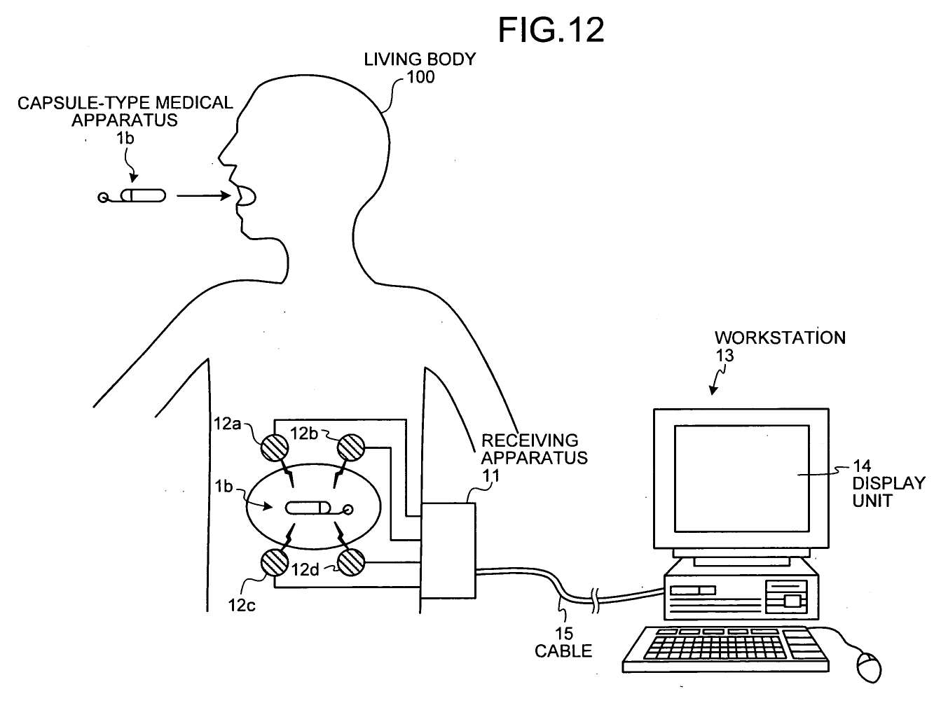

A drug delivery system including the capsule-type medical apparatus 1b according to the second modification of the first embodiment of the present invention will be described. FIG. 12 is a schematic diagram of an exemplary configuration of a drug delivery system including the capsule-type medical apparatus 1b according to the second modification of the first embodiment of the present invention. As shown in FIG. 12, the drug delivery system according to the second modification of the first embodiment of the present invention includes the capsule-type medical apparatus 1b in place of the capsule-type medical apparatus 1a of the drug delivery system according to the first modification of the first embodiment. In other respects, the configuration of the drug delivery system according to the second modification is the same as that of the first modification of the first embodiment, and the same components are denoted by the same reference characters.

In the drug delivery system according to the second modification of the first embodiment, the capsule-type medical apparatus 1b is swallowed by the living body 100 from the mouth together with the drug D1 held by the thread-like drug holding unit 17, and inserted into the interior of the living body 100. Thereafter, the capsule-type medical apparatus 1b moves through the sites in the living body 100 successively or intermittently following the peristaltic movements and the like, while sequentially capturing images as the drug-source information at predetermined intervals. The images as the drug-source information are sequentially radio transmitted to the receiving apparatus 11 outside.

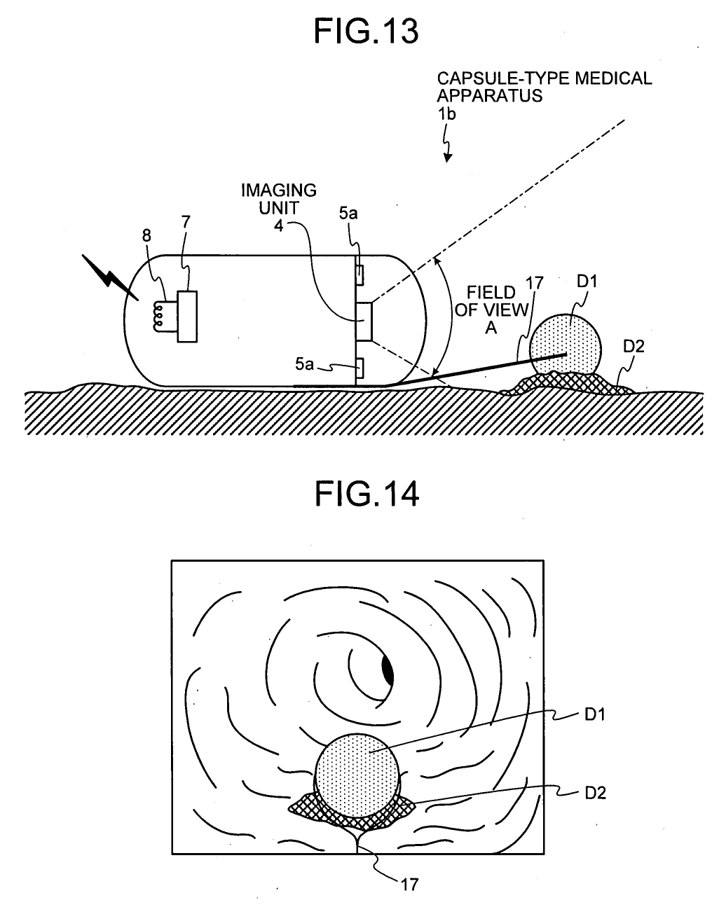

An operation of the capsule-type medical apparatus 1b inserted into the interior of the living body 100 will be described. FIG. 13 is a schematic diagram illustrating a state of the capsule-type medical apparatus 1b according to the second modification of the first embodiment inserted into the interior of the living body. FIG. 14 is a schematic diagram of a specific example of an image captured by the capsule-type medical apparatus 1b according to the second modification of the first embodiment.

As shown in FIG. 13, the capsule-type medical apparatus 1b inserted into the interior of the living body 100 arranges the drug D1 at a position (e.g., a position away from the imaging unit 4 by a distance suitable for the imaging) within the field of view A of the imaging unit 4 according to the function of the drug holding unit 17. The drug D1 arranged at a position within the field of view A is brought into contact with the body fluid in the living body 100 in a substantially similar condition to that when swallowed by the living body 100 by itself, and gradually dissolves into the body fluid. Thus, the drug D1 is released to the site in the living body 100 as the drug solution D2 and gradually decreases. The imaging unit 4 sequentially captures images covering the drug D1 gradually decreasing and the site in the living body around the drug D1 (i.e., the image as the drug-source information) at predetermined intervals.

The image captured by the imaging unit 4 shows the drug D1 (i.e., drug D1 held in the drug holding unit 17) released to the site within the living body 100 as the drug solution D2 and gradually decreasing, and the site around the drug D1 (i.e., site in the living body 100 to which the drug D1 is released). In other words, the imaging unit 4 captures the image as the drug-source information which indicates the release condition of the drug D1 with respect to the site in the living body 100 and the site in the living body 100 where the drug D1 is released.

The series of images as the drug-source information captured by the imaging unit 4 are sequentially radio transmitted through the antenna 8 by the radio communication unit 7, sequentially taken into the workstation 13 via the receiving apparatus 11 and the like, and displayed on the display unit 14 of the workstation 13 in real time similarly to the first modification of the first embodiment.

The series of images as the drug-source information displayed on the display unit 14 in real time show, as shown in FIG. 14, the drug D1 released to the site in the living body 100 and decreases and the site around the drug D1 in the living body 100. Therefore, the doctor or the nurse can confirm in real time the release condition of the drug D1 with respect to the site in the living body 100 and at the same time confirm in real time whether the drug D1 is actually released to a desirable site (i.e., site such as an affected site as a release target of the drug D1) in the living body 100 by visually confirming the series of images as the drug-source information similarly to the first modification of the first embodiment.

As described above, in the second modification of the first embodiment of the present invention, the capsule-type medical apparatus is configured so that the capsule-like casing and the drug are connected by the thread-like connecting member, the image covering the drug connected (held) by the connecting member and the surrounding area of the drug within the field of view is captured, and the image covering the drug which is released to the site in the living body and decreases and the surrounding area is radio transmitted to the receiving apparatus outside the living body. Further, similarly to the first modification of the first embodiment, the capsule-type medical apparatus is configured so that the images received by the receiving apparatus are sequentially displayed on the display unit. Hence, in addition to the advantages and effects of the first modification of the first embodiment, the second modification has an advantage that the drug can be brought into contact with the body fluid in the living body in a substantially similar condition with that of the drug delivered to the interior of the living body by itself. As a result, it is possible to provide the capsule-type medical apparatus and the drug delivery system including the capsule-type medical apparatus which have the advantages and effects of the first modification of the first embodiment, and allow for the confirmation of the release condition (i.e., a state of dissolution of the drug in the body fluid) of the drug released to the site in the living body in a similar state as that of the drug delivered to the interior of the living body by itself.

Further, since the connecting member can be deformed into a desirable shape while maintaining the connected state of the drug and the capsule-like casing, the pains of the living body at the time of insertion of the capsule-type medical apparatus and the drug into the living body can be alleviated.

### Third Modification of First Embodiment

A third modification of the first embodiment of the present invention will be described. In the first embodiment described above, the drug D1 is held in the drug holding unit 3 which is formed like a bag or basket having meshes. In the third modification of the first embodiment, the drug D1 is held in a state sandwiched between plural transparent plates.

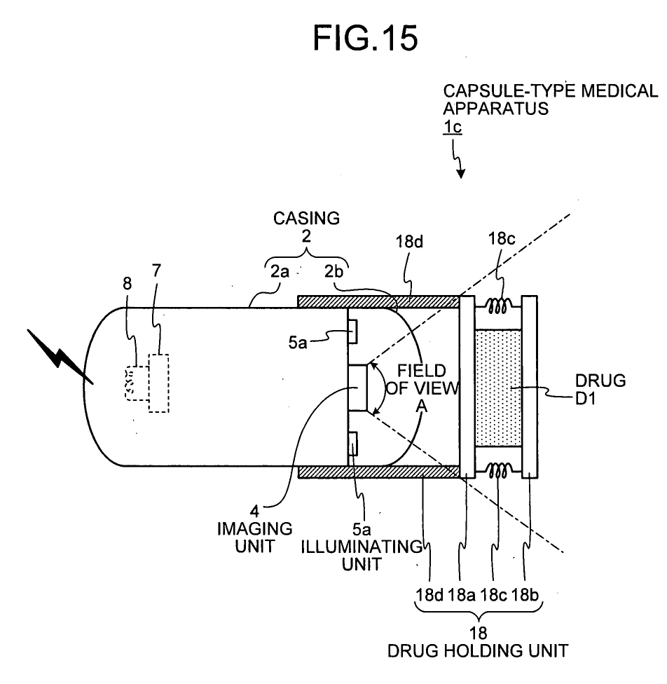

FIG. 15 is a schematic diagram of one exemplary configuration of the capsule-type medical apparatus according to the third modification of the first embodiment of the present invention. As shown in FIG. 15, a capsule-type medical apparatus 1c according to the third modification of the first embodiment has a drug holding unit 18 in place of the drug holding unit 3 of the capsule-type medical apparatus 1 according to the first embodiment described above. In other respects, the configuration of the capsule-type medical apparatus according to the third modification is the same as that of the first embodiment, and the same components are denoted by the same reference characters.

The drug holding unit 18 serves as a holding unit which holds the drug D1 to be delivered to the interior of the living body in a releasable manner with respect to the site in the living body. Specifically, the drug holding unit 18 has two holding plates 18a and 18b that sandwich the drug D1 in a releasable manner with respect to the site in the living body, a spring 18c that generates pressing force of the holding plates 18a and 18b with respect to the drug D1, and a connecting member 18d that connects the holding plate 18a and the casing 2.

The holding plates 18a and 18b formed of a transparent member with high optical transparency sandwich the drug D1 between opposing surfaces thereof to press and hold the drug D1. The holding plates 18a and 18b are in surface contact with the drug D1, and hold the drug D1 without blocking the contact between an outer circumferential portion of the drug D1 and the body fluid in the living body. The drug D1 held by holding plates 18a and 18b as described above contacts with the body fluid in the living body and gradually dissolves in the body fluid from the outer circumferential portion toward a central portion.

Further, being transparent members as mentioned above, the holding plates 18a and 18b hold the drug D1 at a position within the field of view A without blocking the field of view A of the imaging unit 4. Here, the imaging unit 4 captures an image covering the drug D1 and the site in the living body around the drug D1 visible through the holding plates 18a and 18b.

The pressing force of the holding plates 18a and 18b with respect to the drug D1 is generated by the spring 18c. The spring 18c has one end connected to the holding plate 18a and another end connected to the holding plate 18b. The spring 18c connects the holding plates 18a and 18b, and generates the pressing force applied to the drug D1 sandwiched between the holding plates 18a and 18b. The spring 18c serves to apply the elastic force (pressing force) in such a direction that the holding plate 18b is brought closer to the holding plate 18a which is arranged at the side of the casing 2, for example.

The connecting member 18d connects the casing 2 with one of the holding plates 18a and 18b (e.g., the holding plate 18a arranged at the side of the casing 2) that hold the drug D1. The connecting member 18d supports the holding plates 18a and 18b in such a manner that the drug D1 is arranged at a predetermined position within the field of view A of the imaging unit 4.

The drug holding unit 18 configured as described above holds the drug D1 in a releasable manner with respect to the site in the living body and arranges the drug D1 at a substantially fixed position (e.g., suitable position for the imaging of the drug D1 by the imaging unit 4) within the field of view A without blocking the field of view A of the imaging unit 4. The drug D1 held by the drug holding unit 18 gradually dissolves in the body fluid in the living body from the outer circumferential portion toward the central portion, and is gradually released to the site in the living body as the drug solution D2 and decreases. The imaging unit 4 captures an image (i.e., the image as the drug-source information) covering the drug D1 and the site around the drug D1 in the living body visible through the holding plates 18a and 18b of the drug holding unit 18.

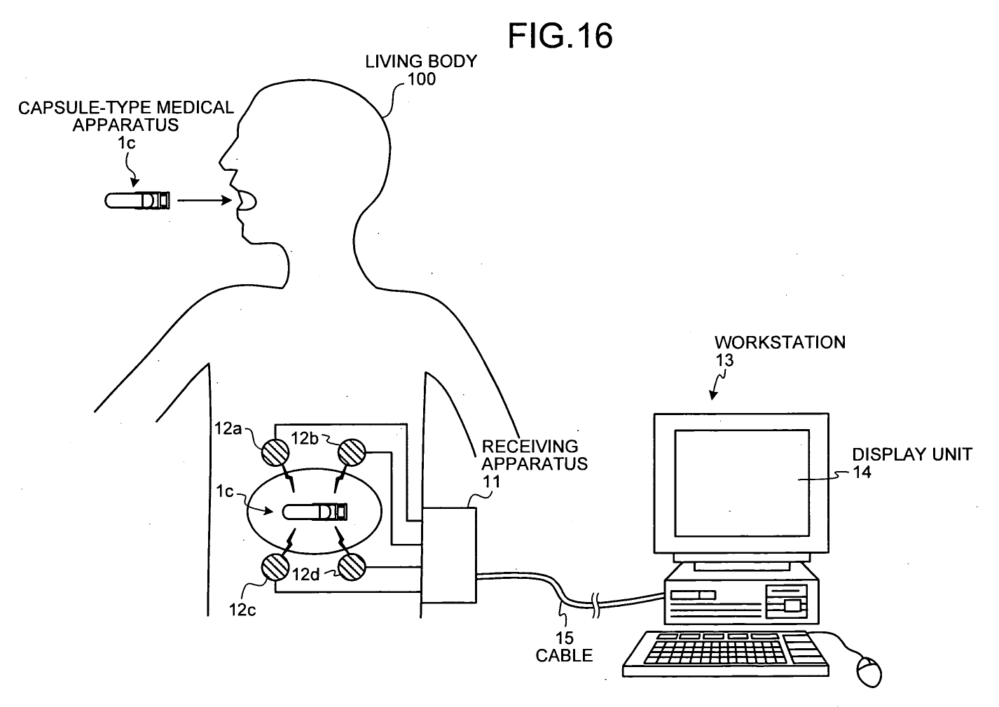

A drug delivery system including the capsule-type medical apparatus 1c according to the third modification of the first embodiment of the present invention will be described. FIG. 16 is a schematic diagram of an exemplary configuration of the drug delivery system including the capsule-type medical apparatus 1c according to the third modification of the first embodiment of the present invention. As shown in FIG. 16, the drug delivery system according to the third modification of the first embodiment of the present invention has the capsule-type medical apparatus 1c in place of the capsule-type medical apparatus 1 of the drug delivery system according to the first embodiment. In other respects, the configuration of the drug delivery system according to the third modification is the same as that of the first embodiment, and the same components are denoted by the same reference characters.

In the drug delivery system according to the third modification of the first embodiment, the capsule-type medical apparatus 1c is swallowed by the living body 100 from the mouth while the drug D1 is sandwiched between the holding plates 18a and 18b, and thus inserted into the interior of the living body 100 together with the drug D1. Thereafter, the capsule-type medical apparatus 1c moves through the sites in the living body 100 successively or intermittently following the peristaltic movements and the like, while sequentially capturing images as the drug-source information at predetermined intervals. The images as the drug-source information are sequentially radio transmitted to the receiving apparatus 11 outside.

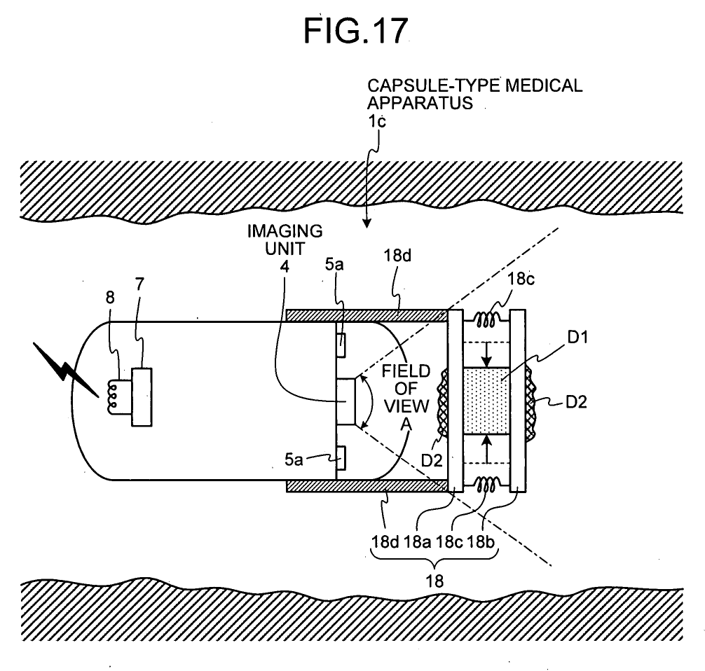

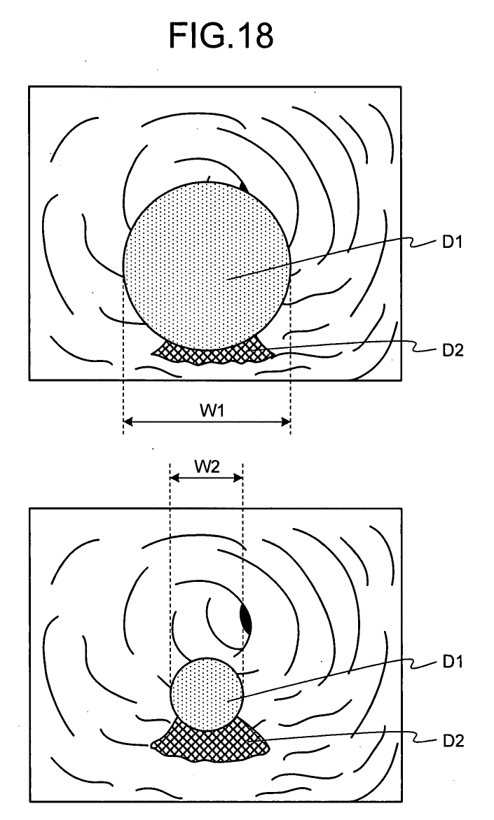

An operation of the capsule-type medical apparatus 1c inserted into the living body 100 will be described. FIG. 17 is a schematic diagram illustrating a state of the capsule-type medical apparatus 1c according to the third modification of the first embodiment inserted into the interior of the living body. FIG. 18 is a schematic diagram of a specific example of an image captured by the capsule-type medical apparatus 1c according to the third modification of the first embodiment.

As shown in FIG. 17, the capsule-type medical apparatus 1c inserted into the living body 100 holds the drug D1 at a fixed position (e.g., suitable position for the imaging) within the field of view A of the imaging unit 4 by the drug holding unit 18, and makes the body fluid in the living body 100 contact with the outer circumferential portion of the drug D1. The drug D1 thus held gradually dissolves into the body fluid in the living body 100 from the outer circumferential portion toward the central portion, and at the same time is released to the site in the living body 100 as the drug solution D2 and gradually decreases. The drug holding unit 18 keeps holding the drug D1 which gradually decreases from the outer circumferential portion towards the central portion at the fixed position within the field of view A. The imaging unit 4 sequentially captures the image (i.e., the image as the drug-source information) covering the drug D1 gradually decreasing from the outer circumferential portion and the site around the drug D1 in the living body through the holding plates 18a and 18b of the drug holding unit 18 at predetermined intervals.

The images captured by the imaging unit 4 show, as shown in FIG. 18, the drug D1 which gradually decreases from the outer circumferential portion in a state held at substantially the fixed position within the field of view A and the surrounding area of the drug D1 (i.e., site where the drug D1 is released in the living body 100). In other words, the imaging unit 4 captures images as the drug-source information indicating the release condition of the drug D1 with respect to the site in the living body 100 and the site in the living body 100 where the drug D1 is released.

A series of images captured by the imaging unit 4 as the drug-source information are sequentially radio transmitted via the antenna 8 by the radio communication unit 7, sequentially taken into the workstation 13 via the receiving apparatus 11 and the like, and displayed in real time on the display unit 14 of the workstation 11, similarly to the first embodiment.

The series of images as the drug-source information displayed in real time on the display unit 14 indicate the drug D1 released to the site in the living body 100 and decreases and the site in the living body 100 around the drug D1 as shown in FIG. 18, for example. Therefore, the doctor or the nurse can confirm in real time the release condition of the drug D1 with respect to the site in the living body 100, and at the same time, can confirm in real time whether the drug D1 is actually released to a desirable site (i.e., site such as an affected site as a release target of the drug D1) in the living body 100 by visually confirming the series of images as the drug-source information, similarly to the first embodiment.

Further, the series of images as the drug-source information show the drug D1 substantially at the fixed position. Therefore, the decreased state of the drug D1 which is released to the site in the living body 100 and decreases (i.e., dissolved state of the drug D1) can be easily confirmed, and the amount of decrease of the drug D1 released as the drug solution D2 and decreases can be easily grasped through visual confirmation of the state of the drug D1 shown in each image of the series of images as the drug-source information. For example, as shown in FIG. 18, the decreased state of the drug D1 in the living body 100 and the actual amount of decrease can be easily known through the comparison between width W1 of the drug D1 shown in the image as the drug-source information and width W2 of the drug D1 shown in the subsequently-captured image as the drug-source information.

As described above, in the third modification of the first embodiment of the present invention, the capsule-type medical apparatus is configured so that the drug is held between the transparent holding plates in such a manner that the contact between the outer circumferential portion of the drug and the body fluid in the living body is not obstructed, the drug held between the holding plates is arranged at substantially the fixed position in the field of view of the imaging unit, the image covering the drug released to the site in the living body and decreases and the surrounding area of the drug is captured through the transparent holding plates, and the images covering the drug and the surrounding area are radio transmitted to the receiving apparatus outside the living body. Further, the capsule-type medical apparatus is configured so as to sequentially display the images received by the receiving apparatus on the display unit similarly to the first embodiment. Hence, in addition to the advantages and effects of the first embodiment, the third modification has an advantage that the series of images which allow for an easy confirmation of the decreased state of the drug which is released to the site in the living body and decreases can be captured. As a result, it is possible to provide a capsule-type medical apparatus and a drug delivery system which have the advantages and effects of the first embodiment and allow for easy grasp of the decreased state and the decreased amount of the drug with respect to the site in the living body.

Further, since the holding plates sandwiching the drug are supported at a fixed position in the field of view of the imaging unit, the drug can be held at a fixed position suitable for the imaging in the field of view until the capsule-type medical apparatus inserted into the interior of the living body together with the drug is excreted outside the living body. As a result, the image covering the drug which is released to the site in the living body and decreases and the surrounding area thereof can be more clearly captured, and the release condition of the drug with respect to the site in the living body can be easily confirmed.

### Second Embodiment

A second embodiment of the present invention will be described. In the first embodiment described above, the image covering the drug D1 and the surrounding area of the drug D1 is captured as the drug-source information. In the second embodiment, the drug solution D2 in which the drug D1 held in the casing dissolves in the body fluid is discharged toward a site in the living body from the casing, and concentration of the drug solution D2 thus discharged is detected as the drug-source information.

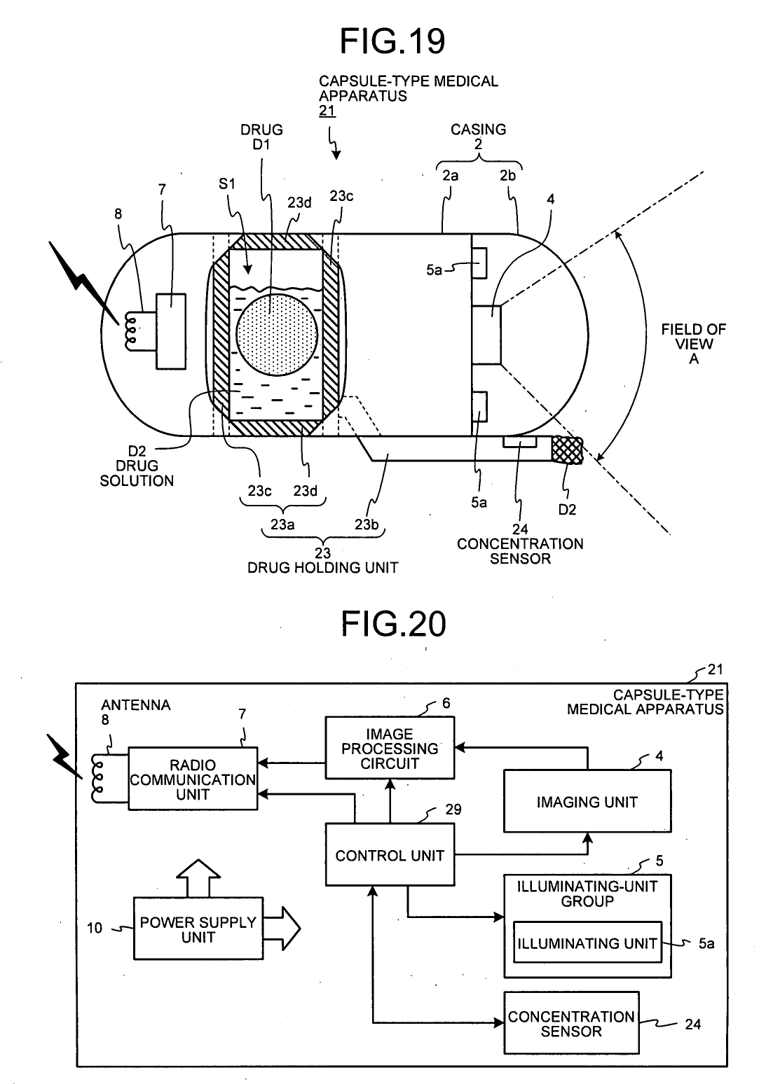

FIG. 19 is a schematic diagram of an exemplary configuration of a capsule-type medical apparatus according to the second embodiment of the present invention. FIG. 20 is a schematic block diagram of an exemplary configuration of the capsule-type medical apparatus according to the second embodiment of the present invention. As shown in FIGS. 19 and 20, a capsule-type medical apparatus 21 according to the second embodiment has a drug holding unit 23, and a control unit 29 in place of the drug holding unit 3 and the control unit 9, respectively, of the capsule-type medical apparatus 1 according to the first embodiment. Further, the capsule-type medical apparatus 21 further includes a concentration sensor 24 that detects the drug-source information mentioned above. In other respects, the configuration of the second embodiment is the same as that of the first embodiment, and the same components are denoted by the same reference characters.

The drug holding unit 23 serves as a holding unit that holds the drug D1 in a releasable manner with respect to the site in the living body. Specifically, the drug holding unit 23 includes a storage unit 23a which holds the drug D1 and stores the drug solution D2 in which the drug D1 dissolves in the body fluid in the living body, and a discharge tube 23b that discharges the drug solution D2 stored in the storage unit 23a.

The storage unit 23a is formed inside the casing 2 and forms a drug holding space S1 to contain the drug D1. Further, the storage unit 23a makes the body fluid in the living body flow from outside the casing 2 into the drug holding space S1, and stores the drug solution D2 in which the drug D1 dissolves in the body fluid in the drug holding space S1. The storage unit 23a is formed with a wall member 23c which separates the drug holding space S1 and an inner space of the casing 2 and a semipermeable membrane 23d which forms a part of an outer wall of the casing 2 (more specifically, the casing main body 2a).

The wall member 23c is one of walls forming the drug holding space S1. The wall member 23c separates the inner space of the casing 2 and the drug holding space S1, and secures liquid-tightness of two spaces. Further, an opening is formed in one portion of the wall member 23c, and one end of the discharge tube 23b is connected to the opening of the wall member 23c. The discharge tube 23b arranged at the wall member 23c communicates with the drug holding space S1.

The semipermeable membrane 23d is one of the walls forming the drug holding space S1, and forms a part of the outer walls of the casing main body 2a. The semipermeable membrane 23d blocks the drug D1 and the drug solution D2, and transmits only the body fluid in the living body. The semipermeable membrane 23d makes the body fluid in the living body flow into the drug holding space S1 due to osmotic pressure, and blocks in/outflow of the drug D1 and the drug solution D2 through the semipermeable membrane 23d.

The discharge tube 23b has one end connected to the opening of the wall member 23c and another end arranged outside the casing 2 (e.g., near the optical dome 2b). The discharge tube 23b communicates with the drug holding space S1 and discharges the drug solution D2 produced inside the drug holding space S1 to the site in the living body (i.e., outside the casing 2).

The concentration sensor 24 serves as a detecting unit that detects the drug-source information indicating the release condition of the drug D1 released to the site in the living body. Specifically, the concentration sensor 24 is arranged near a discharge outlet of the discharge tube 23b and detects the drug concentration of the drug solution D2 which flows from the drug holding space S1 through the discharge tube 23b and is discharged (released) to the site in the living body. The drug solution D2 stored in the drug holding space S1 is produced through dissolution of the drug D1 in the body fluid flowing through the semipermeable membrane 23d from the living body. Hence, the concentration of the drug D1 in the drug solution D2, i.e., the drug concentration of the drug solution D2 corresponds to the amount of decrease of the drug D1 which dissolves in the body fluid and decreases in the drug holding space S1. Thus, the drug concentration of the drug solution D2 serves as the drug-source information which indicates the release condition and the amount of decrease of the drug D1 which is released to the site in the living body as the drug solution D2 and decreases. The concentration sensor 24 detects the drug concentration of the drug solution D2 as the drug-source information. The concentration sensor 24 transmits the detected drug concentration of the drug solution D2, in other words, the drug-source information to the control unit 29.

The control unit 29 controls the driving of the imaging unit 4, the illuminating-unit group 5, the image processing circuit 6, and the radio communication unit 7, substantially similarly to the control unit 9 of the capsule-type medical apparatus 1 according to the first embodiment, and further controls the driving of the concentration sensor 24. The control unit 29 controls the concentration sensor 24 so as to detect the drug concentration of the drug solution D2 discharged from the discharge tube 23b, and controls the illuminating-unit group 5 and the imaging unit 4 in synchronization with the detection process of the drug concentration by the concentration sensor 24.

Based on the control by the control unit 29, the illuminating-unit group 5 illuminates the field of view A of the imaging unit 4, and the imaging unit 4 captures images of a subject located within the field of view A illuminated by the illuminating-unit group 5 in synchronization with the operation of the illuminating-unit group 5. The imaging unit 4 captures images of a site in the living body where the drug solution D2 is discharged from the discharge tube 23b, in other words, the site in the living body where the drug D1 is released as the drug solution D2. The image captured by the imaging unit 4 serves as site information which indicates the site in the living body where the drug D1 is actually released as the drug solution D2. The imaging unit 4 sequentially captures the images as the site information.

The control unit 29 acquires the drug concentration as the drug-source information detected by the concentration sensor 24, and controls the radio communication unit 7 so as to radio transmit the images as the site information captured in synchronization with the detection process of the drug concentration and the drug concentration as the drug-source information in association with each other. Based on the control by the control unit 29, the radio communication unit 7 generates radio signals including the drug concentration as the drug-source information and the images as the site information, and transmits the generated radio signals from the antenna 8. Thus, the radio signals including the drug concentration as the drug-source information and the images as the site information are sequentially transmitted to the outside of the living body.

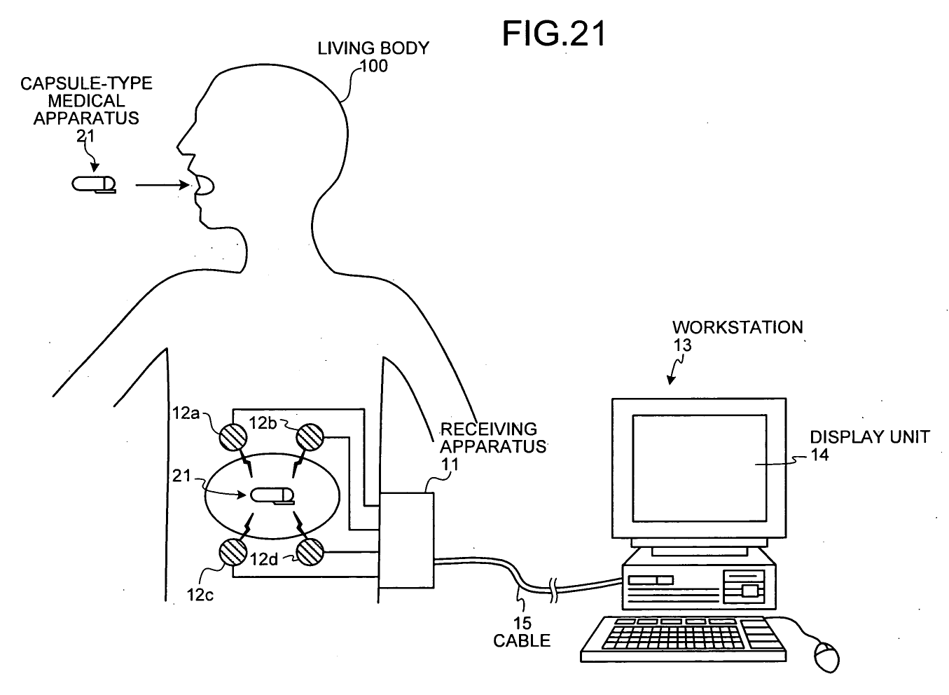

A drug delivery system including the capsule-type medical apparatus 21 according to the second embodiment of the present invention will be described. FIG. 21 is a schematic diagram of an exemplary configuration of the drug delivery system including the capsule-type medical apparatus 21 according to the second embodiment of the present invention. As shown in FIG. 21, the drug delivery system according to the second embodiment of the present invention includes the capsule-type medical apparatus 21 in place of the capsule-type medical apparatus 1 of the drug delivery system according to the first embodiment. In other respects, the configuration of the drug delivery system according to the second embodiment is the same as that of the first embodiment, and the same components are denoted by the same reference characters.

In the drug delivery system according to the second embodiment, the capsule-type medical apparatus 21 is swallowed by the living body 100 from the mouth while holding the drug D1 in the drug holding space S1 of the drug holding unit 23, and is inserted into the interior of the living body 100. The capsule-type medical apparatus 21 moves through the sites in the living body 100 successively or intermittently following the peristaltic movements and the like, and discharges (releases) the drug solution D2 which is a mixture of the drug D1 and the body fluid in the drug holding space S1 to the site in the living body 100. The capsule-type medical apparatus 21 detects the drug concentration of the drug solution D2 (i.e., the drug-source information indicating the release condition and the amount of decrease of the drug D1), and acquires the images of the site inside the living body 100 where the drug solution D2 is discharged (i.e., the site information indicating the site in the living body 100 where the drug D1 is released as the drug solution D2). The capsule-type medical apparatus 21 sequentially acquires the drug concentration as the drug-source information and the images as the site information, and sequentially radio transmits the acquired drug concentration as the drug-source information and the images as the site information.

The receiving apparatus 11 sequentially receives the drug concentration as the drug-source information and the images as the site information from the capsule-type medical apparatus 21 via one of the receiving antennas 12a to 12d. The workstation 13 sequentially takes in the drug concentration as the drug-source information and the images as the site information received by the receiving apparatus 11 via the cable 15, for example, and sequentially displays the drug concentration as the drug-source information and the images as the site information on the display unit 14. Thus, the workstation 13 displays a series of pieces of the drug-source information (drug concentration) and the site information (images) detected by the capsule-type medical apparatus 21 in the living body 100 on the display unit 14 in real time.

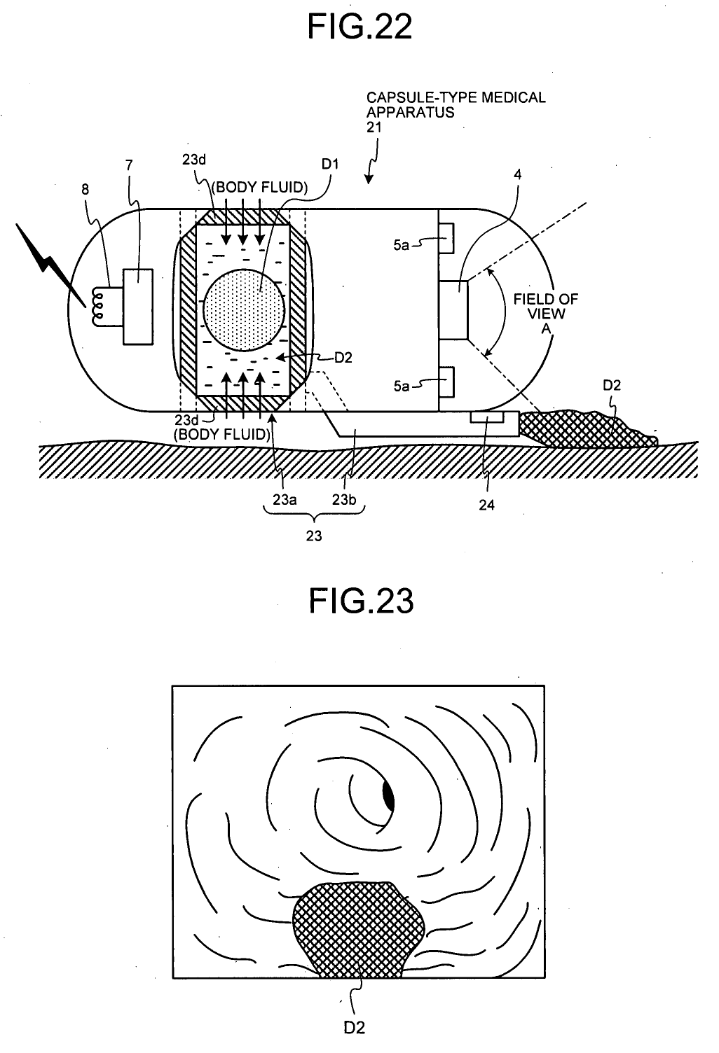

An operation of the capsule-type medical apparatus 21 inserted into the living body 100 will be described. FIG. 22 is a schematic diagram illustrating a state of the capsule-type medical apparatus 21 according to the second embodiment inserted into the living body. FIG. 23 is a schematic diagram of a specific example of an image captured by the capsule-type medical apparatus 21 according to the second embodiment.

As shown in FIG. 22, the capsule-type medical apparatus 21 inserted into the living body 100 makes the body fluid in the living body 100 flow into the storage unit 23a (i.e., into the drug holding space S1) via the semipermeable membrane 23d, and produces the drug solution D2 in which the drug D1 dissolves in the body fluid thus flowing inside in the storage unit 23a. The drug solution D2 in the storage unit 23a thus produced is discharged (released) to the site in the living body 100 after flowing through the discharge tube 23b and discharged from the discharge tube 23b. The drug D1 in the storage unit 23a gradually dissolves in the body fluid flowing into the storage unit 23a via the semipermeable membrane 23d, and is released to the site in the living body 100 as the drug solution D2. Accordingly, the amount of the drug D1 gradually decreases.

When the drug solution D2 is discharged to the site in the living body 100, the concentration sensor 24 detects the drug concentration of the drug solution D2. In synchronization with the detection, the imaging unit 4 captures images of the site in the living body 100 where the drug solution D2 is discharged. The drug concentration detected by the concentration sensor 24 is concentration of the drug D1 contained in the drug solution D2 discharged to the site in the living body 100 from the discharge tube 23b, and is the drug-source information which indicates the release condition and the amount of decrease of the drug D1 which is released to the site in the living body 100 as the drug solution D2 and is decreased. Further, the images captured by the imaging unit 4 is the site information indicating the site in the living body 100 where the drug solution D2 containing the drug D1 is discharged (released) as shown in FIG. 23, for example.

During the period after the capsule-type medical apparatus 21 is inserted into the living body 100 and is naturally excreted outside the living body 100, the concentration sensor 24 sequentially detects the drug concentration as the drug-source information every time the drug solution D2 is discharged from the discharge tube 23b, or every time a predetermined time elapses. In synchronization with the detection process of the concentration sensor 24, the imaging unit 4 sequentially captures the images as the site information. The drug concentration as the drug-source information and the images as the site information are sequentially radio transmitted from the antenna 8 by the radio communication unit 7.

The drug-source information (drug concentration) and the site information (images) sequentially radio transmitted from the capsule-type medical apparatus 21 are sequentially received by the receiving apparatus 11, and sequentially taken into the workstation 13 via the cable 15, for example. Thereafter, the workstation 13 displays the drug concentration as the drug-source information and the images as the site information in association with each other in real time on the display unit 14.

The series of pieces of drug-source information (drug concentration) displayed in real time on the display unit 14 indicate the release condition and the amount of decrease of the drug D1 which is released to the site in the living body 100 as the drug solution D2, and the site information (images) displayed in association with respective pieces of the series of the drug-source information indicate the site in the living body 100 where the drug solution D2 containing the drug D1 is released as shown in FIG. 23, for example. Therefore, the doctor or the nurse can confirm in real time the decreased state of the drug D1 which is released to the site in the living body 100 and decreases and the site (e.g., stomach, duodenum, small intestine, or large intestine) within the living body 100 where the drug D1 is actually released by sequentially visually confirming the drug concentration as the drug-source information and the images as the site information. As a result, the doctor or the nurse can confirm in real time the release condition of the drug D1 with respect to the site in the living body 100, and confirm in real time whether the drug D1 is actually released to a desirable site (i.e., site such as an affected site which is a release target of the drug D1) in the living body 100 even while the capsule-type medical apparatus 21 is in the living body 100.

Further, the visual confirmation of the drug concentration as the drug-source information allows for easily grasping the decreased amount (i.e., the released amount of the drug D1 to the site in the living body 100 as the drug solution D2) of the drug D1 which is difficult to know merely from the images captured by the imaging unit 4 as shown in FIG. 23, for example.

As described above, in the second embodiment of the present invention, the capsule-type medical apparatus is configured so that the drug solution is produced through the dissolution of the drug in the body fluid flowing from inside the living body to the drug holding space holding the drug, the drug solution in the drug holding space is discharged to the site in the living body, the drug concentration of the drug solution is detected, the images of the site in the living body where the drug solution is discharged are captured, and the drug concentration and the images in the living body are radio transmitted to the receiving apparatus outside in association with each other. Further, the capsule-type medical apparatus is configured so as to sequentially display the drug concentration and the image received by the receiving apparatus as a pair on the display unit. Hence, the release condition of the drug to the interior of the living body and the decreased amount of the drug at the discharge, and the site in the living body where the drug is released can be confirmed in real time through the visual confirmation of the pair of drug concentration and the image sequentially displayed on the display unit even while the drug is in the living body. As a result, it is possible to provide a capsule-type medical apparatus and a drug delivery system including the same which allow for real-time confirmation on whether the drug is actually released to the site in the living body and real-time confirmation of the site in the living body where the drug is actually released, and the decreased amount of drug at the discharge.

### Third Embodiment

A third embodiment of the present invention will be described. In the first embodiment, the image covering the drug D1 and the surrounding area of the drug D1 is captured as the drug-source information. In the third embodiment, the drug D1 is held between a light-emitting surface of a light-emitting-element group and a light-receiving surface of a light-receiving element, and the drug-source information indicating the release condition of the drug D1 is detected based on light intensity of light emitted from the light-emitting-element group and received by the light-receiving element.

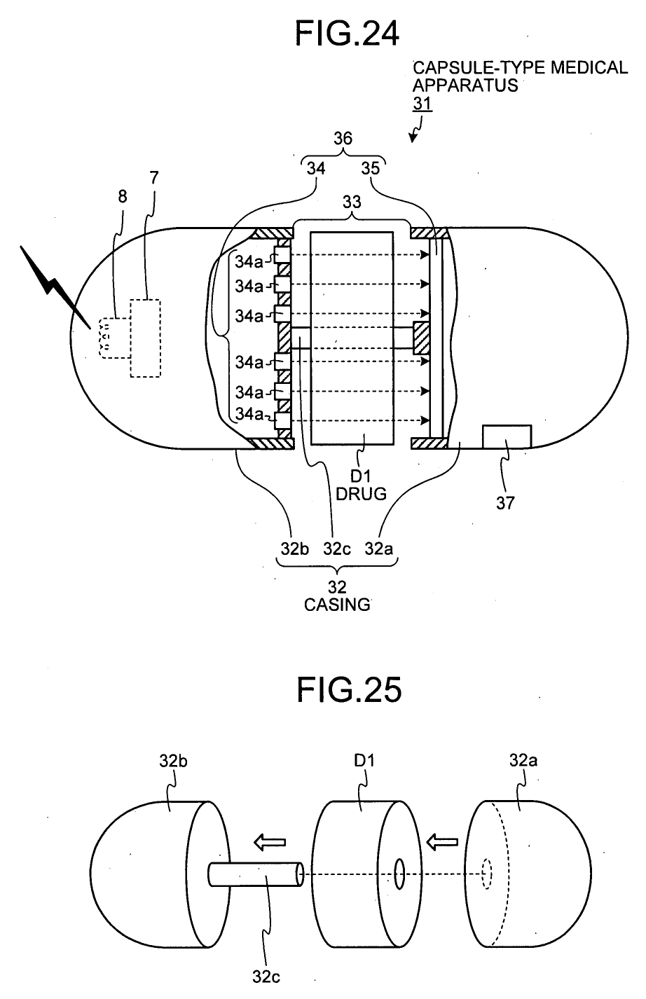

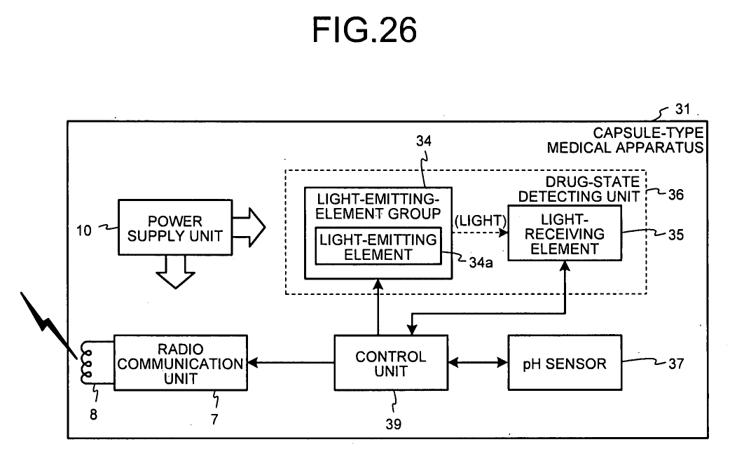

FIG. 24 is a schematic diagram of an exemplary configuration of a capsule-type medical apparatus according to the third embodiment of the present invention. FIG. 25 is a schematic diagram of an example of a disassembled state of the capsule-like casing. FIG. 26 is a schematic block diagram of an exemplary configuration of the capsule-type medical apparatus according to the third embodiment.

The capsule-type medical apparatus according to the third embodiment detects light intensity as the drug-source information using the light-emitting-element group and the light-receiving element arranged so as to sandwich the drug D1 therebetween instead of acquiring the image as the drug-source information as in the capsule-type medical apparatus 1 according to the first embodiment, and further detects pH value of body fluid indicating the site in the living body where the drug D1 is released as the site information.

As shown in FIGS. 24 to 26, a capsule-type medical apparatus 31 according to the third embodiment includes a casing 32 in which a drug holding space 33 is formed to hold the drug D1, a drug-state detecting unit 36 which detects the state of the drug D1, a pH sensor 37 which detects pH value of the body fluid in the living body, and a control unit 39 which controls the driving of each component of the capsule-type medical apparatus 31. The drug-state detecting unit 36 is configured with a light-emitting-element group 34 and a light-receiving element 35 that are arranged so as to oppose with each other with the drug D1 placed therebetween. The capsule-type medical apparatus 31 further includes, similarly to the capsule-type medial apparatus 1 according to the first embodiment, the radio communication unit 7, the antenna 8, and the power supply unit 10 which supplies driving power to each component of the capsule-type medical apparatus 31.

The casing 32 is a capsule-shaped casing formed in a suitable size for the insertion into the living body. The casing 32 houses each component of the capsule-type medical apparatus 31, such as the drug-state detecting unit 36, the pH sensor 37, the control unit 39, the radio communication unit 7, the antenna 8, and the power supply unit 10. The casing 32 forms the drug holding space 33 that holds the drug D1. Further, the casing 32 is formed with partial casings 32a and 32b that sandwich the drug D1 therebetween, and a connecting member 32c that connects the partial casings 32a and 32b. The casing 32 can be easily swallowed by the living body from the mouth, and easily move through the digestive tract of the living body following the peristaltic movements and the like.

The partial casings 32a and 32b are formed as two divided portions of the capsule-like casing 32, and are connected by the connecting member 32c. Specifically, the partial casing 32b is a light-emitting-side partial casing in which the light-emitting-element group 34 is arranged, whereas the partial casing 32a is a light-receiving-side partial casing in which the light-receiving element 35 is arranged. The partial casings 32a and 32b are arranged in such a manner that each light-emitting surface of the light-emitting-element group 23 opposes to the light-receiving surface of the light-receiving element 35, and are connected by the connecting member 32c.

The connecting member 32c serves as a connecting unit that connects the partial casings 32a and 32b, and also serves as a holding unit that holds the drug D1 between the partial casings 32a and 32b. Specifically, the connecting member 32c connects the partial casings 32a and 32b by penetrating a through hole formed at a central portion of the drug D1 as shown in FIG. 25, for example. The partial casings 32a and 32b connected by the connecting member 32c place each light-emitting surface of the light-emitting-element group 34 opposite to the light-receiving surface of the light-receiving element 35, and form the drug holding space 33 sandwiched between the light-emitting-element group 34 and the light-receiving element 35. The connecting member 32c holds the drug D1 in the drug holding space 33 sandwiched between the partial casings 32a and 32b. The drug D1 is held in a releasable manner with respect to the site in the living body by the connecting member 32c.

The drug-state detecting unit 36 is configured with the light-emitting-element group 34 and the light-receiving element 35 that oppose with each other sandwiching the drug D1 held in the drug holding space 33, and detects the state of the drug D1. The drug-state detecting unit 36 serves as a detecting unit that optically detects the drug-source information indicating the release condition of the drug D1 to the site in the living body.

Specifically, the light-emitting-element group 34 has plural light-emitting elements 34a. The plural light-emitting elements 34a are realized with LEDs, for example, and are arranged to the partial casing 32b opposite to the light-receiving surface of the light-receiving element 35 with the drug D1 therebetween. Here, the plural light-emitting elements 34a are arranged on the surface opposite to the light-receiving surface of the light-receiving element 35 in a column-like shape, a cross-like shape, or a matrix-like shape. The plural light-emitting elements 34a emit light of predetermined intensity to the drug D1 or the light-receiving surface of the light-receiving element 35 according to the release condition of the drug D1 to the site in the living body. The light emitted by each of the plural light-emitting elements 34a is blocked by the drug D1 when the drug D1 in the drug holding space 33 has not substantially been released to the site in the living body. Thereafter, as the drug D1 is released to the site in the living body and decreases, the number of light-emitting elements 34a that directly oppose to the light-receiving element 35 increases. Therefore, the light intensity of light from the light-emitting-element group 34 received by the light-receiving element 35 gradually increases along with the decrease of the drug D1.

The light-receiving element 35 is realized with a photodiode or a CCD, for example, and is arranged to the partial casing 32a so as to oppose to each light-emitting surface of the light-emitting-element group 34 across the drug D1. The light-receiving element 35 receives the light emitted from each light-emitting element 34a of the light-emitting-element group 34, and detects a light-receiving area (i.e., received light intensity) of the light received from the light-emitting element 34a. The light-receiving element 35 changes the light-receiving area of the light received from the light-emitting-element group 34 according to the release condition of the drug D1 to the site in the living body. Specifically, the light-receiving area of the light-receiving element 35, i.e., the received light intensity of the light-receiving element 35 gradually increases along with the release and decrease of the drug D1 in the drug holding space 33 to the site in the living body. Therefore, the received light intensity of the light-receiving element 35 corresponds to the decreased amount of the drug D1 which is released to the site in the living body and decreases. Therefore, the received light intensity of the light-receiving element 35 serves as the drug-source information that indicates the release condition and the decreased amount of the drug D1 which is released as the drug solution D2 to the site in the living body and decreases. The light-receiving element 35 detects the received light intensity of the light emitted from the light-emitting-element group 34 as the drug-source information. The light receiving element 35 transmits the detected received light intensity, i.e., the drug-source information to the control unit 39.

The pH sensor 37 serves as a site detecting unit that detects the site in the living body where the drug D1 is released as the drug solution D2. Specifically, the pH sensor 37 is arranged near an outer wall surface of the partial casing 32a, for example, to detect pH value of the body fluid in the living body. The pH value of the body fluid varies depending on the sites in the living body. For example, pH value of the body fluid takes a value indicating strong acid in the stomach, whereas takes a value indicating neutral in the small intestine. Therefore, the pH sensor 37 detects the pH value of the body fluid in the living body as the site information indicating the site in the living body where the drug D1 is released as the drug solution D2. The pH sensor 37 transmits detected pH values, i.e., the site information to the control unit 39.

The control unit 39 controls the driving of each of the light-emitting element 34a of the light-emitting-element group 34, the light-receiving element 35, the pH sensor 37, and the radio communication unit 7. The control unit 39 controls each of the light-emitting elements 34a so as to emit light every time a predetermined time elapses, and controls the light-receiving element 35 so as to detect the received light intensity as the drug-source information. In synchronization therewith, the control unit 39 controls the pH sensor 37 so as to detect the pH value as the site information.

Based on the control by the control unit 39, the plural light-emitting elements 34a emit light at predetermined intervals, and the light-receiving element 35 sequentially detects the received light intensity as the drug-source information at predetermined intervals, and sequentially transmits the detected received light intensity as the drug-source information to the control unit 39. In synchronization therewith, the pH sensor 37 sequentially detects the pH value as the site information, and sequentially transmits the detected pH value to the control unit 39 as the site information.

The control unit 39 acquires the received light intensity detected by the light-receiving element 35 as the drug-source information, and acquires the pH value detected by the pH sensor 37 as the site information. The control unit 39 controls the radio communication unit 7 so as to radio transmit the drug concentration as the drug-source information and the pH value as the site information in association with each other. Based on the control by the control unit 39, the radio communication unit 7 generates radio signals including the received light intensity as the drug-source information and the pH value as the site information, and transmits the generated radio signals from the antenna 8. Thus, the radio signals including the received light intensity as the drug-source information and the pH value as the site information are sequentially transmitted to the outside of the living body.

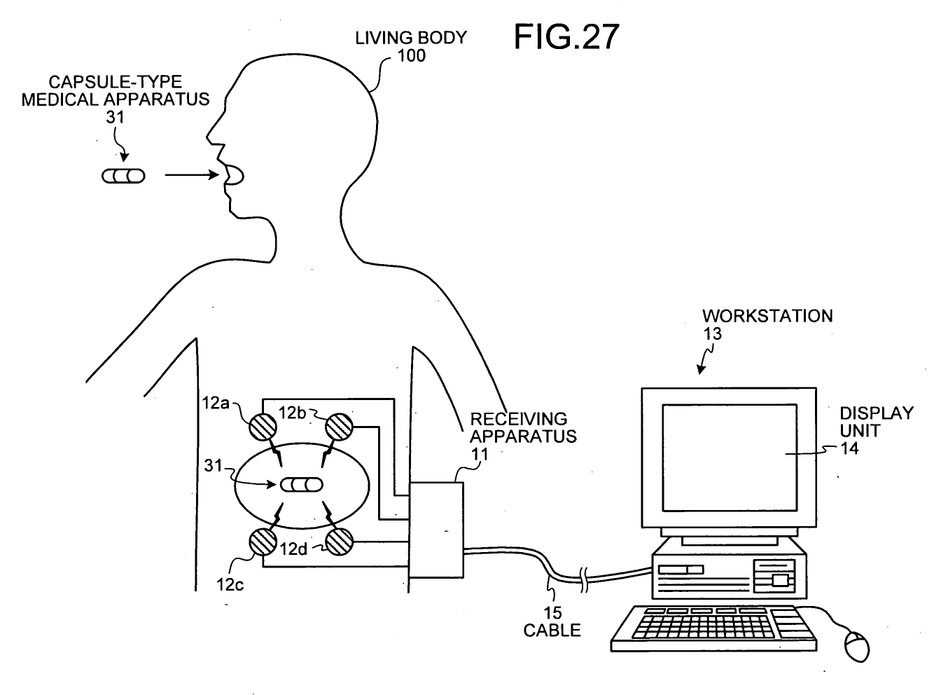

A drug delivery system including the capsule-type medical apparatus 31 according to the third embodiment of the present invention will be described. FIG. 27 is a schematic diagram of an exemplary configuration of the drug delivery system including the capsule-type medical apparatus 31 according to the third embodiment of the present invention. As shown in FIG. 27, the drug delivery system according to the third embodiment of the present invention includes the capsule-type medical apparatus 31 in place of the capsule-type medical apparatus 1 of the drug delivery system according to the first embodiment. In other respects, the configuration of the drug delivery system according to the third embodiment is the same as that of the first embodiment, and the same components are denoted by the same reference characters.

In the drug delivery system according to the third embodiment, the capsule-type medical apparatus 31 is swallowed by the living body 100 from the mouth while holding the drug D1 in the drug holding space 33 and is inserted into the living body 100. The capsule-type medical apparatus 31 moves through the sites in the living body 100 successively or intermittently following peristaltic movements and the like, while releasing the drug D1 in the drug holding space 33 to the site in the living body 100 as the drug solution D2. The capsule-type medical apparatus 31 detects the received light intensity (i.e., the drug-source information indicating the release condition and the decreased amount of the drug D1) of the light-receiving element 35 which increases along with the decrease of the drug D1 at predetermined intervals, and at the same time, detects the pH value (i.e., the site information indicating the site where the drug D1 is released in the living body 100) of the body fluid at the site in the living body 100 where the drug D1 is released as the drug solution D2. The capsule-type medical apparatus 31 sequentially acquires the received light intensity as the drug-source information and the pH value as the site information, and sequentially radio transmits the acquired received light intensity as the drug-source information and the pH value as the site information.

The receiving apparatus 11 sequentially receives the received light intensity as the drug-source information and the pH value as the site information from the capsule-type medical apparatus 31 via one of the receiving antennas 12a to 12d. The workstation 13 sequentially takes in the received light intensity as the drug-source information and the pH value as the site information received by the receiving apparatus 11 via the cable 15, for example, and sequentially displays the received light intensity as the drug-source information and the pH value as the site information on the display unit 14. Thus, the workstation 13 displays in real time a series of pieces of drug-source information (received light intensity of the light-receiving element 35) and the site information (pH value of the body fluid in the living body) detected by the capsule-type medical apparatus 31 in the living body 100.

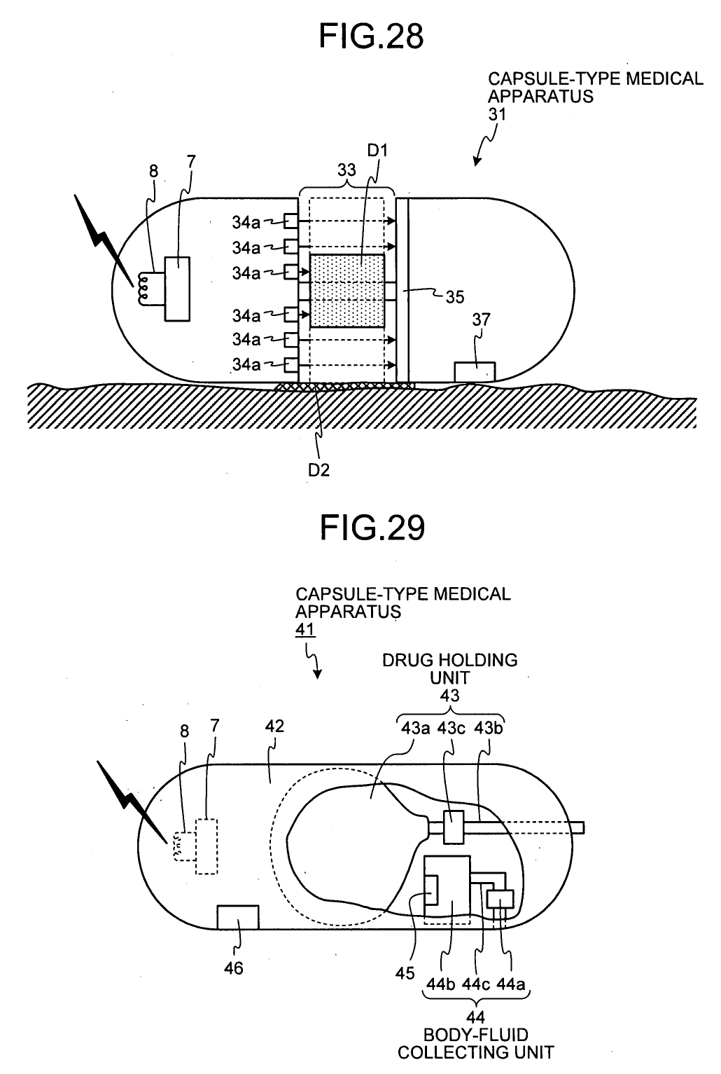

An operation of the capsule-type medical apparatus 31 inserted into the living body 100 will be described. FIG. 28 is a schematic diagram illustrating a state of the capsule-type medical apparatus 31 according to the third embodiment inserted into the living body. As shown in FIG. 28, the capsule-type medical apparatus 31 inserted into the living body 100 holds the drug D1 in the drug holding space 33, and brings the drug D1 into contact with the body fluid in the living body 100. The drug D1 gradually dissolves in the body fluid in the living body 100 from the outer circumferential portion to the central portion, and is released to the site in the living body 100 as the drug solution D2 and gradually decreases.

Each of the plural light-emitting elements 34a emits light at predetermined intervals to the drug D1 which is released and gradually decreasing or the light-receiving surface of the light-receiving element 35. The light-receiving element 35 receives the light not blocked by the drug D1 of the light emitted by the plural light-emitting elements 34a to detect the received light intensity of the received light. In synchronization with the detection, the pH sensor 37 detects the pH value of the body fluid at the site in the living body 100 where the drug D1 is released as the drug solution D2.

The received light intensity detected by the light-receiving element 35 increases along with the decrease of the drug D1 held in the drug holding space 33, and is the drug-source information indicating the release condition and the decreased amount of the drug D1 which is released to the site in the living body 100 as the drug solution D2 and decreases. The pH value detected by the pH sensor 37 is the site information indicating the site in the living body 100 where the drug D1 is released as the drug solution D2.

The light-receiving element 35 sequentially detects the received light intensity as the drug-source information every time a predetermined time elapses during the period after the capsule-type medical apparatus 31 is inserted into the living body 100 until naturally excreted outside the living body 100. In synchronization with the detection process of the light-receiving element 35, the pH sensor 37 sequentially detects the pH value as the site information. The received light intensity as the drug-source information and the pH value as the site information are sequentially radio transmitted from the antenna 8 by the radio communication unit 7.

The drug-source information (received light intensity of light received by the light-receiving element 35) and the site information (pH value of the body fluid) sequentially radio transmitted from the capsule-type medical apparatus 31 are sequentially received by the receiving apparatus 11, and sequentially taken into the workstation 13 via the cable 15, for example. Thereafter, the received light intensity as the drug-source information and the pH value as the site information are displayed on the display unit 14 of the workstation 13 in association with each other in real time.

The series of pieces of drug-source information (received light intensity of the light received by the light-receiving element 35) displayed in real time on the display unit 14 indicate the release condition and the decreased amount of the drug D1 released to the site in the living body 100 as the drug solution D2. The site information (pH value of the body fluid) displayed in association with respective pieces of the series of drug-source information indicates the site in the living body 100 where the drug D1 is released. The doctor or the nurse can confirm in real time the decreased amount of the drug D1 which is released to the site in the living body and decreases, and the site (e.g., stomach, duodenum, small intestine, or large intestine) in the living body 100 where the drug D1 is actually released by sequentially and visually confirming the received light intensity as the drug-source information and the pH value as the site information. As a result, the doctor or the nurse can confirm in real time the release condition of the drug D1 to the site in the living body 100, and also confirm in real time whether the drug D1 is actually released to a desirable site (i.e., site such as an affected site as a release target of the drug D1) in the living body 100 or not even while the capsule-type medical apparatus 31 is in the living body 100.

As described above, the capsule-type medical apparatus according to the third embodiment of the present invention is configured so that the drug is held between the light-emitting surface of the light-emitting-element group and the light-receiving surface of the light-receiving element opposing with each other, the received light intensity of the light-receiving element which increases along with the decrease in the drug is detected, the pH value of the body fluid at the site in the living body where the drug is released is detected, and the detected received light intensity of the light-receiving element and the pH value of the body fluid are radio transmitted to the receiving apparatus outside in association with each other. Further, the capsule-type medical apparatus is configured so that the pairs of received light intensities and pH values received by the receiving apparatus are sequentially displayed on the display unit. Hence, it is possible to confirm the release condition of the drug to the living body, the decreased amount of drug at the time of release, and the site in the living body where the drug is released can be confirmed in real time through the visual confirmation of the pairs of received light intensities and the pH values of the body fluid sequentially displayed on the display unit even while the drug is inside the living body. As a result, it is possible to provide a capsule-type medical apparatus and a drug delivery system including the same in a simple configuration so as to allow for real-time confirmation of whether the drug is actually released to the site in the living body, and real-time confirmation of the site in the living body where the drug is actually released and the decreased amount of the drug at the time of release.

### Fourth Embodiment

A fourth embodiment of the present invention will be described. In the first embodiment, the image covering the drug D1 and the surrounding area of the drug D1 is captured as the drug-source information. In the fourth embodiment, the body fluid in the living body is collected, and the drug-source information indicating the release condition of the drug to the site in the living body is detected based on the collected body fluid.

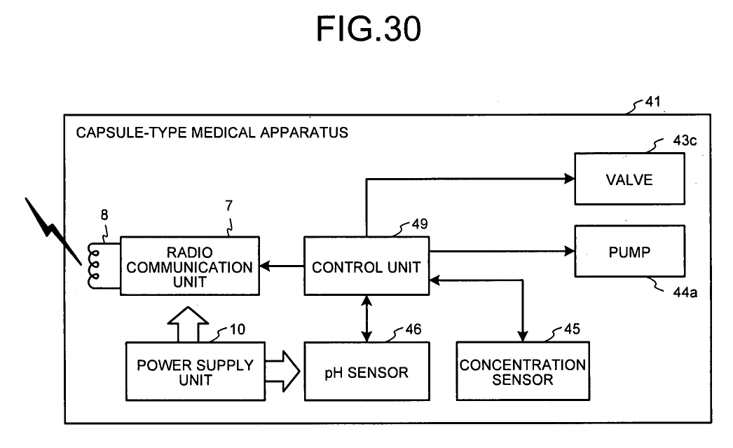

FIG. 29 is a schematic diagram of an exemplary configuration of a capsule-type medical apparatus according to the fourth embodiment of the present invention. FIG. 30 is a schematic block diagram of an exemplary configuration of the capsule-type medical apparatus according to the fourth embodiment of the present invention. A capsule-type medical apparatus 41 according to the fourth embodiment collects the body fluid at the site in the living body instead of acquiring the image as the drug-source information as in the capsule-type medical apparatus 1 according to the first embodiment, to detect the drug-source information indicating the release condition of the drug to the site in the living body based on the collected body fluid. Further, the capsule-type medical apparatus 41 detects the pH value of the body fluid as the site information indicating the site in the living body where the drug is released.

As shown in FIGS. 29 and 30, the capsule-type medical apparatus 41 includes a capsule-like casing 42, a drug holding unit 43 which holds and discharges a liquid drug D3, a body-fluid collecting unit 44 which collects the body fluid in the living body, a concentration sensor 45 which detects concentration of bacteria, for example, in the body fluid collected by the body-fluid collecting unit 44, a pH sensor 46 which detects the pH value of the body fluid in the living body, and a control unit 49 which controls the driving of each component of the capsule-type medical apparatus 41. Further, the capsule-type medical apparatus 41 includes, similarly to the capsule-type medical apparatus 1 according to the first embodiment, the radio communication unit 7, the antenna 8, and the power supply unit 10 that supplies the driving power to each component of the capsule-type medical apparatus 41.

The casing 42 is a capsule-like casing formed in a suitable size for the insertion into the living body, and houses respective components of the capsule-type medical apparatus 41, such as the drug holding unit 43, the body-fluid collecting unit 44, the concentration sensor 45, the pH sensor 46, the control unit 49, the radio communication unit 7, the antenna 8, and the power supply unit 10. The casing 42 can be easily swallowed by the living body from the mouth, for example, and can easily move inside the digestive tract of the living body following the peristaltic movements and the like.

The drug holding unit 43 serves as a holding unit that holds the liquid drug D3, and also serves as a drug-discharging unit that discharges (releases) the drug D3 to the site in the living body. Specifically, the drug holding unit 43 includes a balloon 43a that holds the drug D3 and discharges the drug D3 according to contraction force of itself, an discharge tube 43b that channels the drug D3 discharged from the balloon 43a to the outside of the casing 42 (i.e., the site in the living body), and a valve 43c that adjusts a communicated state between the balloon 43a and the discharge tube 43b.

The balloon 43a is realized with an elastic member such as rubber. When the liquid drug D3 is injected to the balloon 43a, the balloon 43a expands and stores the drug D3 inside maintaining the expanded state. The balloon 43a works to discharge the contained drug D3 according to the contraction force of itself latent in the expanded state.

The discharge tube 43b has one end connected to the balloon 43a and another end inserted into an opening of the casing 42. The discharge tube 43b communicates an interior of the balloon 43a (i.e., an internal space where the drug D3 is held) with the outside of the casing 42 when the valve 43c is driven to be open, and releases the drug D3 discharged from the balloon 43a to the outside of the casing 42, i.e., to the site in the living body.

The valve 43c adjusts the communicated state of the balloon 43a and the discharge tube 43b. Specifically, the valve 43c communicates the balloon 43a with the discharge tube 43b when driven to open under the control of the control unit 49. The balloon 43a applies pressure to the drug D3 by its own contraction force and discharges the drug D3. The drug D3 discharged from the balloon 43a passes through the valve 43c and the discharge tube 43b, so as to be released to the site in the living body. On the other hand, the valve 43c blocks the communication between the balloon 43a and the discharge tube 43b when driven to be closed under the control of the control unit 49. The balloon 43a then stops the discharge operation of the drug D3.

The body-fluid collecting unit 44 collects the body fluid in the living body to detect the drug-source information indicating the release condition of the drug D3 to the site in the living body. Specifically, the body-fluid collecting unit 44 includes a pump 44a which sucks (collects) the body fluid from the site in the living body, a body-fluid storage unit 44b which stores the body fluid sucked by the pump 44a, and a suction tube 44c which channels the body fluid sucked by the pump 44a to the body-fluid storage unit 44b.

The pump 44a sucks (collects) the body fluid in the site in the living body under the control of the control unit 49. The suction tube 44c has one end connected to the body-fluid storage unit 44b and another end inserted into an opening of the casing 42. The suction tube 44c channels the body fluid sucked by the pump 44a to the body-fluid storage unit 44b. The body-fluid storage unit 44b obtains the body fluid in the living body through the suction tube 44c and stores the obtained body fluid.

The concentration sensor 45 serves as a detecting unit that detects the drug-source information indicating the release condition of the drug D3 with respect to the site in the living body based on the body fluid collected in the living body by the body-fluid collecting unit 44. Specifically, the concentration sensor 45 is provided in the body-fluid storage unit 44b, for example, so as to detect the concentration of bacteria in the body fluid (i.e., bacteria concentration in the body fluid) in the living body stored in the body-fluid storage unit 44b. When the drug D3 is released to the site in the living body from the drug holding unit 43, for example, the body fluid in the site in the living body is sterilized, and the bacteria concentration in the body fluid decreases. The body-fluid collecting unit 44 collects the body fluid of such a state in the living body. In other words, when the drug D3 is released to the site in the living body from the drug holding unit 43, the body-fluid storage unit 44b stores the body fluid whose bacteria concentration is decreased due to sterilizing effect of the drug D3. The bacteria concentration of the body fluid in the body-fluid storage unit 44b corresponds to the decreased amount of the drug D3 which is discharged by the balloon 43a and decreases. Therefore, the bacteria concentration of the body fluid serves as the drug-source information indicating the release condition of the drug D3 which is released from the balloon 43a to the site in the living body and decreases. The concentration sensor 45 detects the bacteria concentration in the body fluid as the drug-source information. The concentration sensor 45 transmits the detected bacteria concentration of the body fluid, i.e., the drug-source information to the control unit 49.

The pH sensor 46 serves as a site detecting unit that detects the site in the living body where the drug D3 is released. Specifically, the pH sensor 46 is provided near the outer wall surface of the casing 42, for example, and detects the pH value of the body fluid in the living body. The pH value of the body fluid varies according to the site in the living body, as mentioned above. The pH sensor 46 detects the pH value of the body fluid in the living body as the site information indicating the site in the living body where the drug D3 is released. The pH sensor 46 transmits the detected pH value, i.e., the site information to the control unit 49.

The control unit 49 controls the driving of each of the valve 43c of the drug holding unit 43, the pump 44a of the body-fluid collecting unit 44, the concentration sensor 45, the pH sensor 46, and the radio communication unit 7. The control unit 49 controls to drive the opening/closing of the valve 43c every time a predetermined time elapses, for example. In synchronization with the open-driving of the valve 43c, the control unit 49 controls the pH sensor 46 to detect the pH value as the site information. Then, the control unit 49 controls the pump 44a to suck (collect) the body fluid in the living body. Thereafter, the control unit controls the concentration sensor to detect the bacteria concentration of the body fluid as the drug-source information.

Based on the control by the control unit 49, the valve 43c repeats the open/close driving at predetermined intervals. The pH sensor 46 sequentially detects the pH value of the body fluid in the living body in synchronization with the open-driving of the valve 43c and sequentially transmits the obtained pH value as the site information to the control unit 49. The balloon 43a discharges the drug D3 to the site in the living body at predetermined intervals based on the open/close driving of the valve 43c. Further, based on the control by the control unit 49, the pump 44a sucks the body fluid in the living body into the body-fluid storage unit 44b, and the concentration sensor 45 sequentially detects the bacteria concentration of the body fluid stored in the body-fluid storage unit 44b and sequentially transmits the obtained bacteria concentration (i.e., drug-source information) of the body fluid to the control unit 49.

The control unit 49 obtains the bacteria concentration detected by the concentration sensor 45 as the drug-source information and the pH value detected by the pH sensor 46 as the site information. The control unit 49 controls the radio communication unit 7 so as to radio transmit the obtained bacteria concentration as the drug-source information and the pH value as the site information in association with each other. Based on the control by the control unit 49, the radio communication unit 7 generates the radio signals including the bacteria concentration as the drug-source information and the pH value as the site information, and transmits the generated radio signals from the antenna 8. The radio signals including the bacteria concentration as the drug-source information and the pH value as the site information are sequentially transmitted to the outside of the living body.

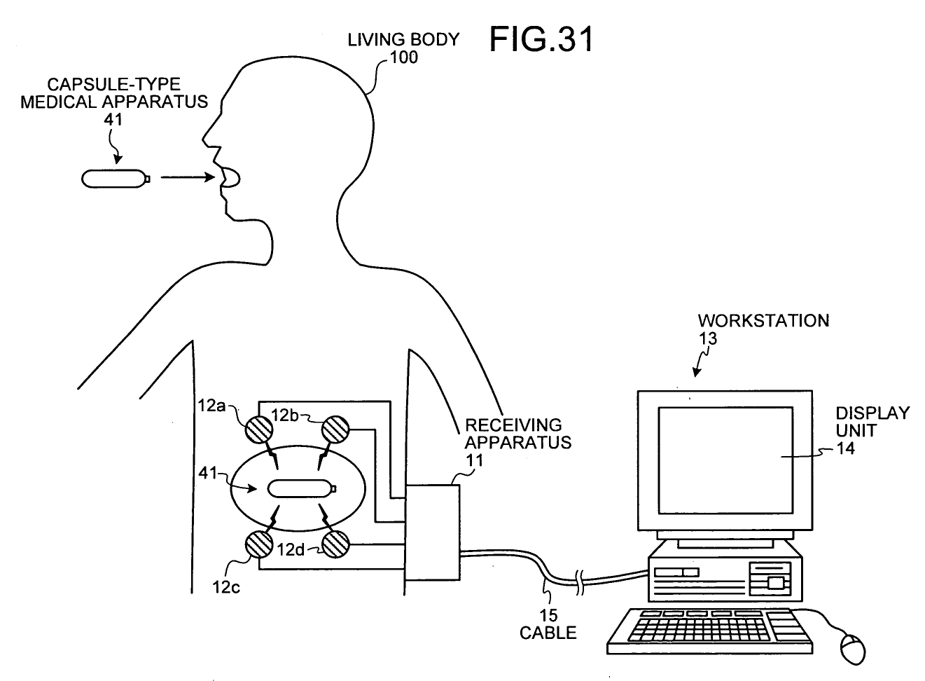

A drug delivery system including the capsule-type medical apparatus 41 according to the fourth embodiment of the present invention will be described. FIG. 31 is a schematic diagram of an exemplary configuration of the drug delivery system including the capsule-type medical apparatus 41 according to the fourth embodiment of the present invention. As shown in FIG. 31, the drug delivery system according to the fourth embodiment of the present invention includes the capsule-type medical apparatus 41 in place of the capsule-type medical apparatus 1 of the drug delivery system according to the first embodiment. In other respects, the configuration of the drug delivery system according to the fourth embodiment is the same with that of the first embodiment, and the same components are denoted by the same reference characters.

In the drug delivery system according to the fourth embodiment, the capsule-type medical apparatus 41 is swallowed by the living body 100 from the mouth while the liquid drug D3 is held in the balloon 43a, and inserted into the living body 100. The capsule-type medical apparatus 41 moves through the sites in the living body 100 successively or intermittently following the peristaltic movements or the like, and discharges (releases) the drug D3 in the balloon 43a to the site in the living body 100 at predetermined intervals. The capsule-type medical apparatus 41 detects the pH value (i.e., the site information indicating the site in the living body 100 where the drug D3 is released) of the body fluid at the site in the living body. Further, the capsule-type medical apparatus 41 collects the body fluid at the site in the living body 100 where the drug D3 is released, and detects the bacteria concentration (i.e., the drug-source information indicating the release condition of the drug D3) of the collected body fluid. The capsule-type medical apparatus 41 sequentially acquires the bacteria concentration as the drug-source information and the pH value as the site information, and sequentially radio transmit the acquired bacteria concentration as the drug-source information and the acquired pH value as the site information.

The receiving apparatus 11 sequentially receives the bacteria concentration as the drug-source information and the pH value as the site information from the capsule-type medical apparatus 41 via one of the receiving antennas 12a to 12d. The workstation 13 sequentially takes in the bacteria concentration as the drug-source information and the pH value as the site information as received by the receiving apparatus 11 via the cable 15, for example, and sequentially displays the bacteria concentration as the drug-source information and the pH value as the site information on the display unit 14. Thus, the workstation 13 displays in real time a series of pieces of drug-source information (i.e., bacteria concentration of the collected body fluid) and the site information (i.e., pH value of the body fluid at the site in the living body) as detected by the capsule-type medical apparatus 41 in the living body 100 on the display unit 14.

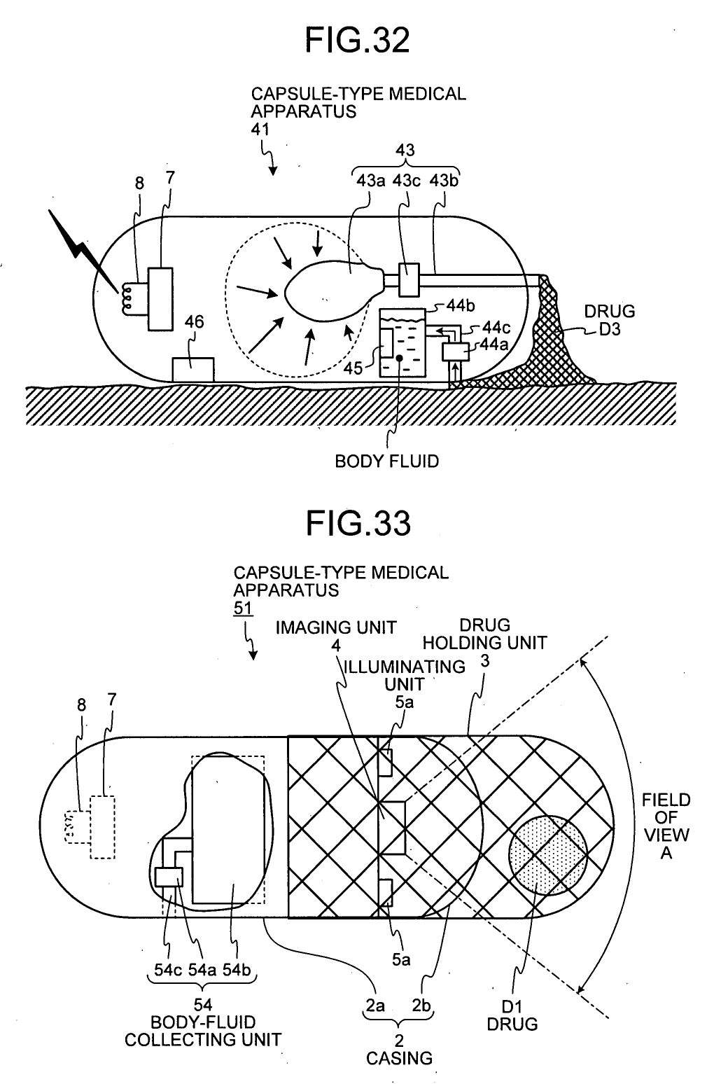

An operation of the capsule-type medical apparatus 41 in the living body 100 will be described. FIG. 32 is a schematic diagram illustrating a state of the capsule-type medical apparatus 41 according to the fourth embodiment inserted into the living body. As shown in FIG. 32, the capsule-type medical apparatus 41 inserted into the living body 100 discharges the drug D3 held in the balloon 43a according to the open-driving of the valve 43c at predetermined intervals. The drug D3 discharged from the balloon 43a passes through the discharge tube 43b and the valve 43c, and is released to the site in the living body 100. The drug D3 in the balloon 43a is released to the site in the living body 100 and gradually decreases. Further, at the site in the living body 100 where the drug D3 is released, the bacteria concentration of the body fluid is decreased due to the sterilizing effect of the drug D3.

In synchronization with the open-driving of the valve 43c, the pH sensor 46 detects the pH value of the body fluid at the site in the living body 100 where the drug D3 is released. The pH value detected by the pH sensor 46 is the site information indicating the site in the living body 100 where the drug D3 is released. The pH sensor 46 sequentially detects the pH value as the site information in synchronization with the open-driving of the valve 43c.

On the other hand, when the drug D3 is released to the site in the living body 100 as described above, the pump 44a sucks the body fluid in the living body 100 which is sterilized by the drug D3. The body fluid sucked by the pump 44a passes through the suction tube 44c and is stored in the body-fluid storage unit 44b. The bacteria concentration of the body fluid stored in the body-fluid storage unit 44b is low due to the sterilizing effect of the drug D3. The concentration sensor 45 detects the bacteria concentration of the body fluid stored in the body-fluid storage unit 44b. The bacteria concentration detected by the concentration sensor 45 is the bacteria concentration of the body fluid sterilized by the drug D3 released from the balloon 43a to the site in the living body 100, and is the drug-source information indicating the release condition of the drug D3 which is released to the site in the living body 100 and decreases.

During the period after the capsule-type medical apparatus 41 is inserted into the living body 100 until naturally excreted outside the living body 100, the concentration sensor 45 sequentially detects the bacteria concentration as the drug-source information every time the drug D3 is released to the site in the living body 100, in other words, every time the valve 43c is driven to be open. The bacteria concentration as the drug-source information and the pH value as the site information are sequentially radio transmitted from the antenna 8 by the radio communication unit 7.

The drug-source information (i.e., bacteria concentration of the collected body fluid) and the site information (i.e., pH value of the body fluid at the site in the living body) sequentially radio transmitted from the capsule-type medical apparatus 41 are sequentially received by the receiving apparatus 11 as described above, and sequentially taken into the workstation 13 via the cable 15, for example. Thereafter, the bacteria concentration as the drug-source information and the pH value as the site information are displayed in real time on the display unit 14 of the workstation 13 in association with each other.

The series of pieces of the drug-source information (i.e., bacteria concentration) displayed in real time on the display unit 14 indicate the release condition of the drug D3 released to the site in the living body 100, whereas the site information (i.e., pH values) displayed in association with respective pieces of the drug-source information indicates the site in the living body 100 where the drug D3 is released. Therefore, the doctor or the nurse can confirm in real time the decreased state of the drug D3 which is released to the site in the living body 100 and decreases and the site (e.g., stomach, duodenum, small intestine, or large intestine) in the living body 100 where the drug D3 is actually released by sequentially and visually confirming the bacteria concentration as the drug-source information and the pH value as the site information. As a result, the doctor or the nurse can confirm the release condition of the drug D3 with respect to the site in the living body 100 in real time and also confirm whether the drug D3 is actually released to a desirable site (i.e., site such as an affected site as a release target of the drug D3) in the living body 100 in real time even while the capsule-type medical apparatus 41 is in the living body 100.

As described above, the capsule-type medical apparatus according to the fourth embodiment is configured so that the liquid drug held in the drug holding unit is released to the site in the living body, the body fluid at the site of the living body where the drug is released is collected, the drug-source information (e.g. bacteria concentration of the collected body fluid) indicating the release condition of the drug to the site in the living body is detected based on the collected body fluid, the pH value (i.e., the site information indicating the site in the living body) of the body fluid at the site in the living body where the drug is released is detected, and the drug-source information and the site information are radio transmitted to the receiving apparatus outside in association with each other. Further, the capsule-type medical apparatus is configured so that the pairs of the drug-source information (bacteria concentration) and the site information (pH value) received by the receiving apparatus are sequentially displayed on the display unit. Therefore, even while the drug is in the living body, it is possible to confirm the release condition of the drug to the living body and the site in the living body where the drug is released in real time through the visual confirmation of the pairs of the drug-source information and the site information sequentially displayed on the display unit. As a result, it is possible to provide a capsule-type medical apparatus and a drug delivery system including the capsule-type medical apparatus which allow for real-time confirmation of whether the drug is actually released to the site in the living body, and real-time confirmation of the site in the living body where the drug is actually released.

Further, since the body fluid in the living body is collected and stored in the body-fluid storage unit in the capsule-type medical apparatus according to the fourth embodiment, symptom of the interior of the living body and a condition of the bacteria can be grasped in detail through the collection and analysis of the body fluid stored in the body-fluid storage unit.

### Fifth Embodiment

A fifth embodiment of the present invention will be described. A capsule-type medical apparatus according to the fifth embodiment has the same configuration as that of the capsule-type medical apparatus 1 according to the first embodiment. In addition, the capsule-type medical apparatus of the fifth embodiment includes a body-fluid collecting unit that collects the body fluid in the living body. Further, a drug delivery system according to the fifth embodiment has the same configuration as that of the drug delivery system according to the first embodiment, and further includes an analyzing apparatus that analyzes the body fluid inside the living body collected by the capsule-type medical apparatus.

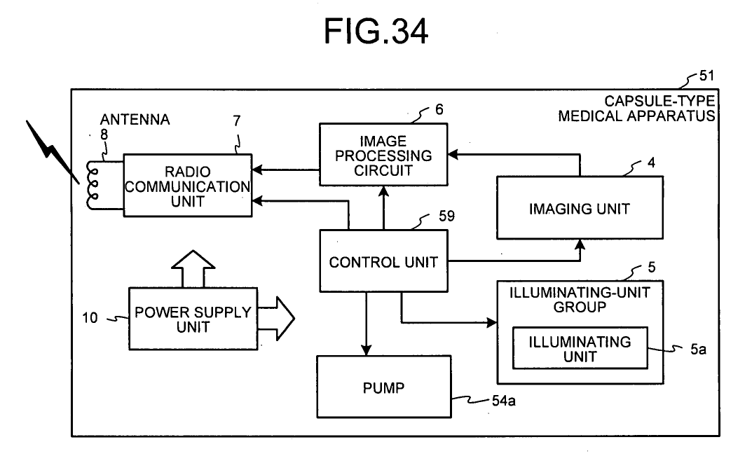

FIG. 33 is a schematic diagram of an exemplary configuration of the capsule-type medical apparatus according to the fifth embodiment of the present invention. FIG. 34 is a schematic block diagram of an exemplary configuration of the capsule-type medical apparatus according to the fifth embodiment of the present invention. As shown in FIGS. 33 and 34, a capsule-type medical apparatus 51 according to the fifth embodiment includes a control unit 59 in place of the control unit 9 of the capsule-type medical apparatus 1 according to the first embodiment, and further includes a body-fluid collecting unit 54 that collects the body fluid in the living body. In other respects, the configuration of the capsule-type medical apparatus according to the fifth embodiment is the same as that of the first embodiment, and the same components are denoted by the same reference characters.

The body-fluid collecting unit 54 serves as a body-fluid collecting unit that collects the body fluid in the living body based on the control by the control unit 59. Specifically, the body-fluid collecting unit 54 includes a pump 54a which sucks (collects) the body fluid from the site in the living body, a body-fluid storage unit 54b which stores the body fluid sucked by the pump 54a, and a suction tube 54c that channels the body fluid sucked by the pump 54a to the body-fluid storage unit 54b.

The pump 54a sucks (collects) the body fluid at an optional site in the living body based on the control by the control unit 59. The suction tube 54c has one end connected to the body-fluid storage unit 54b and another end inserted into the opening of the casing 2 (more specifically the casing main body 2a). The suction tube 54c channels the body fluid sucked by the pump 54a to the body-fluid storage unit 54b. The body-fluid storage unit 54b acquires the body fluid in the living body through the suction tube 54c and stores the obtained body fluid.

The control unit 59 has a similar function to the control unit 9 of the capsule-type medical apparatus 1 according to the first embodiment. In addition, the control unit 59 controls the driving of the pump 54a of the body-fluid collecting unit 54. The control unit 59 controls the driving of the pump 54a at desirable timing set in advance, for example. Based on the control by the control unit 59, the pump 54a sucks (collects) the body fluid at an optional site (e.g., a desirable site where the drug D1 is released) in the living body into the body-fluid storage unit 54b.

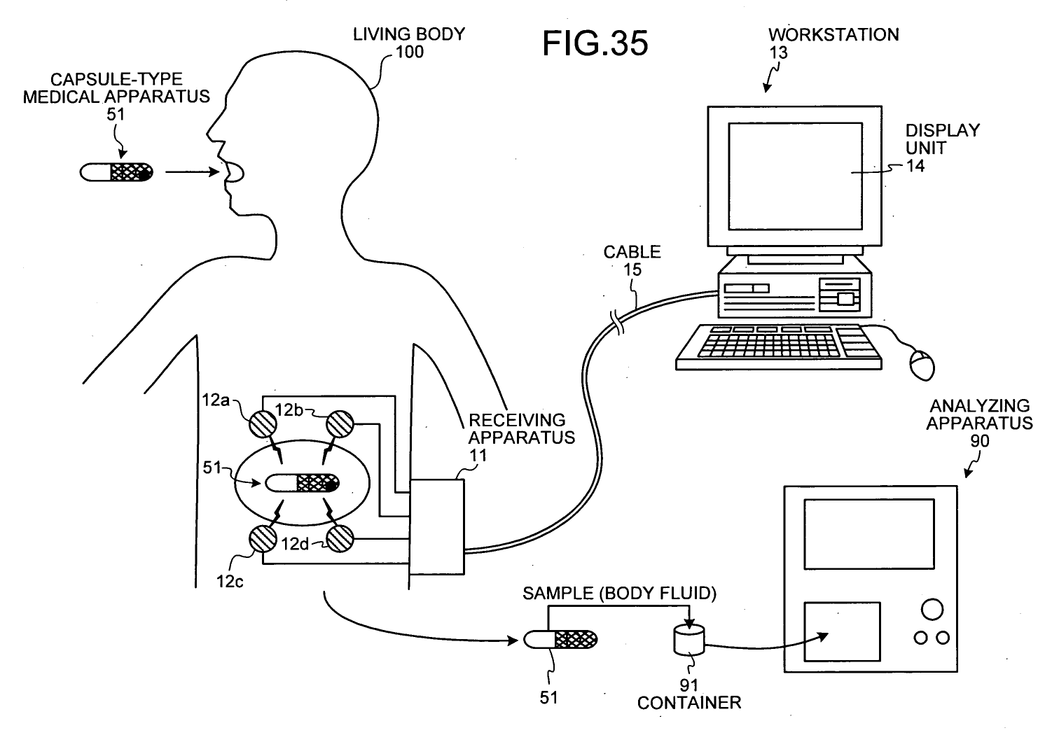

The drug delivery system including the capsule-type medical apparatus 51 according to the fifth embodiment of the present invention will be described. FIG. 35 is a schematic diagram of an exemplary configuration of the drug delivery system including the capsule-type medical apparatus 51 according to the fifth embodiment of the present invention. As shown in FIG. 35, the drug delivery system according to the fifth embodiment of the present invention includes the capsule-type medical apparatus 51 in place of the capsule-type medical apparatus 1 of the drug delivery system according to the first embodiment, and further includes an analyzing apparatus 90 which analyzes the body fluid in the living body 100 collected by the capsule-type medical apparatus 51. In other respects, the configuration of the drug delivery system according to the fifth embodiment is the same as that of the first embodiment, and the same components are denoted by the same reference characters.

In the drug delivery system according to the fifth embodiment, the capsule-type medical apparatus 51 is swallowed by the living body 100 from the mouth, moves through the sites in the living body successively or intermittently following the peristaltic movements or the like, and releases the drug D1, similarly to the capsule-type medical apparatus 1 according to the first embodiment. At the same time, the capsule-type medical apparatus 51 in the living body 100, similarly to the capsule-type medical apparatus 1, sequentially captures the images as the drug-source information and sequentially radio transmits the captured images as the drug-source information to the receiving apparatus 11 outside.

On the other hand, the capsule-type medical apparatus 51 in the living body 100 collects the body fluid at an optional site in the living body 100 and stores the collected body fluid of the living body 100. Thereafter, the capsule-type medical apparatus 51 storing the body fluid is naturally excreted outside the living body 100. The capsule-type medical apparatus 51 naturally excreted from the living body 100 is collected, and the body fluid of the living body 100 stored in the capsule-type medical apparatus 51 is analyzed by the analyzing apparatus 90.

The analyzing apparatus 90 analyzes a sample from inside the living body collected into an adjunctive container 91. Specifically, the body fluid in the living body 100 is collected from the body-fluid storage unit 54b of the capsule-type medical apparatus 51 naturally excreted from the living body 100, and the collected body fluid from the living body 100 is injected into the container 91. The container 91 in which the body fluid from the living body 100 is injected is placed into the analyzing apparatus 90. The analyzing apparatus 90 analyzes the sample (i.e., the body fluid in the living body 100) in the container 91. The analyzing apparatus 90 outputs symptom inside the living body 100, efficacy and effect of the drug D1 delivered to the living body 100, and a condition of bacteria inside the living body 100 as a result of analysis of the body fluid in the living body 100.

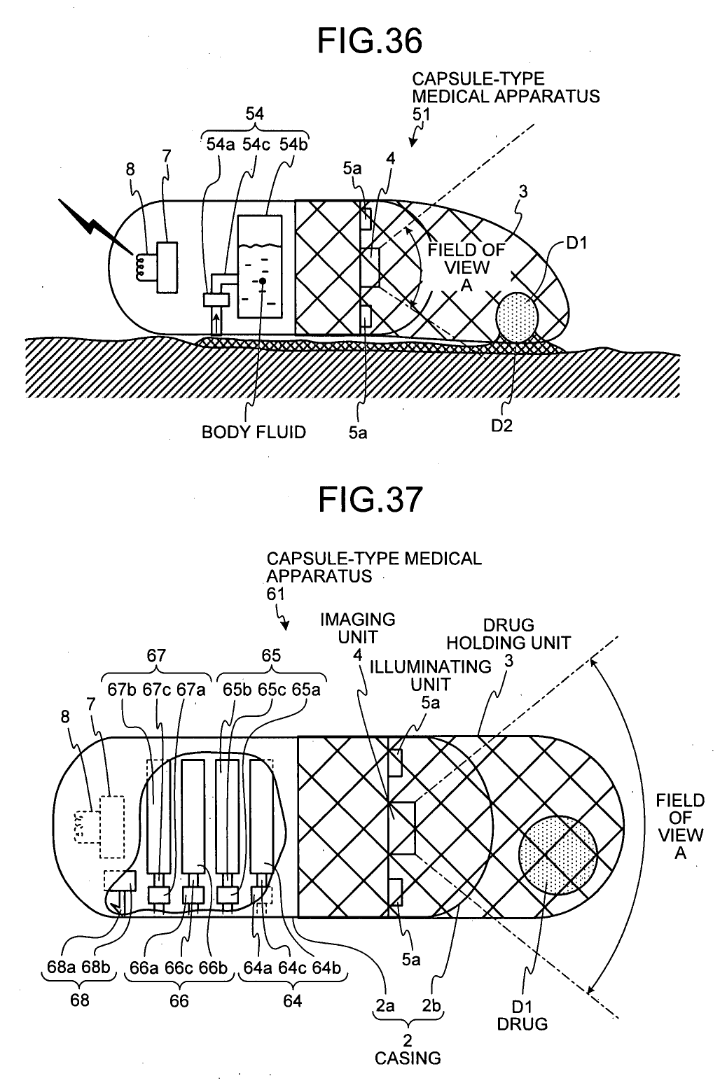

An operation of the capsule-type medical apparatus 51 collecting the body fluid in the living body 100 will be described. FIG. 36 is a schematic diagram of the capsule-type medical apparatus 51 according to the fifth embodiment collecting the body fluid in the living body. As shown in FIG. 36, the capsule-type medical apparatus 51 inserted into the living body 100 moves through the sites in the living body 100 successively or intermittently following the peristaltic movements or the like, and thereafter collects the body fluid at an optional site in the living body 100.

Specifically, the body-fluid collecting unit 54 collects the body fluid at an optional site in the living body 100 based on the control by the control unit 59. The pump 54a sucks the body fluid (e.g., the body fluid at the site where the drug D1 is released) at the optional site in the living body 100. The body fluid sucked by the pump 54a passes through the suction tube 54c and is stored in the body-fluid storage unit 54b. The body-fluid storage unit 54b holds the body fluid collected from the living body 100 until the body fluid is collected into the container 91 as mentioned above.

As described above, the capsule-type medical apparatus according to the fifth embodiment is configured so that the same function and configuration as those of the first embodiment are provided, the body fluid of an optional site in the living body is collected, and the collected body fluid is stored, and the body fluid collected from the living body is analyzed. Therefore, in addition to the advantages and the effects of the first embodiment, the fifth embodiment has an advantage that the body fluid at the site in the living body where the drug is released can be collected and the body fluid at the site where the drug is released can be analyzed. As a result, it is possible to provide a capsule-type medical apparatus and a drug delivery system including the same that have the advantages and the effects of the first embodiment, and allow for acquisition of living-body-related medical information such as a symptom of an interior of a living body, an effect (or efficacy) of a drug, and a condition of bacteria.

### Sixth Embodiment

A sixth embodiment of the present invention will be described. In the fifth embodiment, the body fluid is collected at one optional site in the living body. In the sixth embodiment, plural body-fluid collecting units are provided in the capsule-type medical apparatus, and the body fluid is collected at one optional site in the living body plural times, and the collected body fluid is stored in each of plural body-fluid storage unit separately.

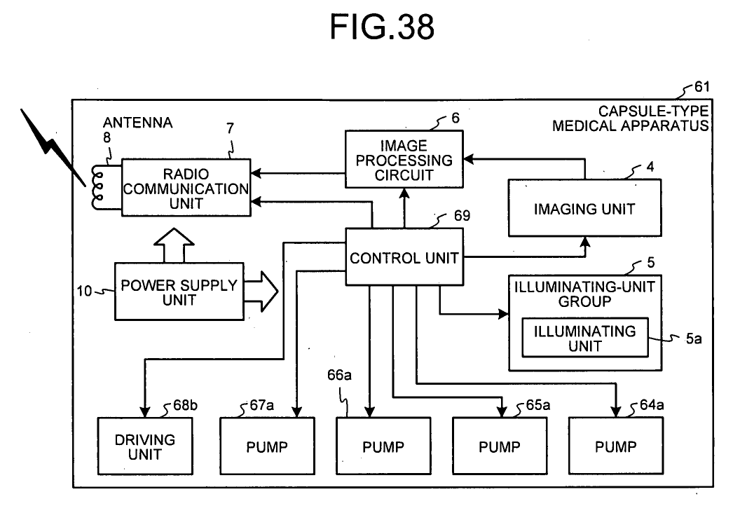

FIG. 37 is a schematic diagram of an exemplary configuration of a capsule-type medical apparatus according to the sixth embodiment. FIG. 38 is a schematic block diagram of one exemplary configuration of the capsule-type medical apparatus according to the sixth embodiment. As shown in FIGS. 37 and 38, a capsule-type medical apparatus 61 according to the sixth embodiment includes plural body-fluid collecting units 64 to 67 and a control unit 69 in place of the body-fluid collecting unit 54 and the control unit 59 of the capsule-type medical apparatus 51 according to the fifth embodiment, respectively. The capsule-type medical apparatus 61 further includes a detainment unit 68 for detaining the capsule-type medical apparatus 61 at one optional site in the living body. In other respects, the configuration of the sixth embodiment is the same as that of the fifth embodiment, and the same components are denoted by the same reference characters.

The plural body-fluid collecting units 64 to 67 serve to collect the body fluid at one optional site in the living body plural times. Each of the body-fluid collecting units 64 to 67 is configured substantially similarly to the body-fluid collecting unit 54 of the capsule-type medical apparatus 51 according to the fifth embodiment. Specifically, the body-fluid collecting unit 64 has a pump 64a, a body-fluid storage unit 64b, and a suction tube 64c; the body-fluid collecting unit 65 has a pump 65a, a body-fluid storage unit 65b, and a suction tube 65c; the body-fluid collecting unit 66 has a pump 66a, a body-fluid storage unit 66b, and a suction tube 66c; and the body-fluid collecting unit 67 has a pump 67a, a body-fluid storage unit 67b, and a suction tube 67c. As far as there are plural body-fluid collecting units as exemplified by the body-fluid collecting units 64 to 67 in the capsule-type medical apparatus 61, the number of the body-fluid collecting units is not limited to four.

The pumps 64a to 67a suck (collect) the body fluid at one optional site in the living body plural times under the control of the control unit 69. The suction tubes 64c to 67c channel the body fluid in the living body sucked by the pumps 64a to 67a into the body-fluid storage units 64b to 67b, respectively. The suction tube 64c has one end connected to the body-fluid storage unit 64b and another end inserted into the opening of the casing main body 2a; the suction tube 65c has one end connected to the body-fluid storage unit 66b and another end inserted into the opening of the casing main body 2a; and the suction tube 67c has one end connected to the body-fluid storage unit 67b and another end inserted into the opening of the casing main body 2a.

The body-fluid storage units 64b to 67b store the body fluid sucked (collected) from one optional site in the living body at plural times separately. Specifically, the body-fluid storage unit 64b stores the body fluid sucked by the pump 64a; the body-fluid storage unit 65b stores the body fluid sucked by the pump 65a; the body-fluid storage unit 66b stores the body fluid sucked by the pump 66a; and the body-fluid storage unit 67b stores the body fluid sucked by the pump 67a.

The detainment unit 68 serves to detain the capsule-type medical apparatus 61 at one optional site in the living body where the body fluid is collected by the body-fluid collecting units 64 to 67. Specifically, the detainment unit 68 is arranged near the outer wall surface of the casing main body 2a, and has a hook 68a which is stuck into one site in the living body and engaged therewith, and a driving unit 68b which pushes out the hook 68a.

The hook 68a is projected outside the casing main body 2a through the opening formed in the casing main body 2a. The hook 68a is stuck into the site in the living body and detains the capsule-type medical apparatus 61 at the site. The driving unit 68b is realized with an elastic member or the like, for example, which serves to push the hook 68a outside. The driving unit 68b detains the hook 68a in the casing main body 2a, and releases the hook 68a under the control of the control unit 69. For example, the driving unit 68b pushes the hook 68a outside the casing main body 2a using an elastic force of the elastic member. Thus, the driving unit 68b sticks the hook 68a into the site in the living body.

The control unit 69 has substantially similar functions to that of the control unit 59 of the capsule-type medical apparatus 51 according to the fifth embodiment. The control unit 69 has substantially similar function to that of the control unit 9 of the capsule-type medical apparatus 1 according to the first embodiment, and in addition, controls the driving of each of the pumps 64a to 67a, and the driving unit 68b. The control unit 69 controls the driving unit at a desirable timing previously set, for example, and thereafter sequentially controls the pumps 64a to 67a every time a predetermined time elapses.

Based on the control by the control unit 69, the driving unit 68b sticks the hook 68a into an optional site in the living body (e.g., a desirable site where the drug D1 is released). Thereafter, the pumps 64a to 67a sequentially suck (collect) the body fluid from the optional site in the living body where the capsule-type medical apparatus 61 is detained by the hook 68a every predetermined time. Thus, moieties of the body fluid sucked by the pumps 64a to 67a at predetermined time intervals for plural times are stored in the plural body-fluid storage units 64b to 67b separately.

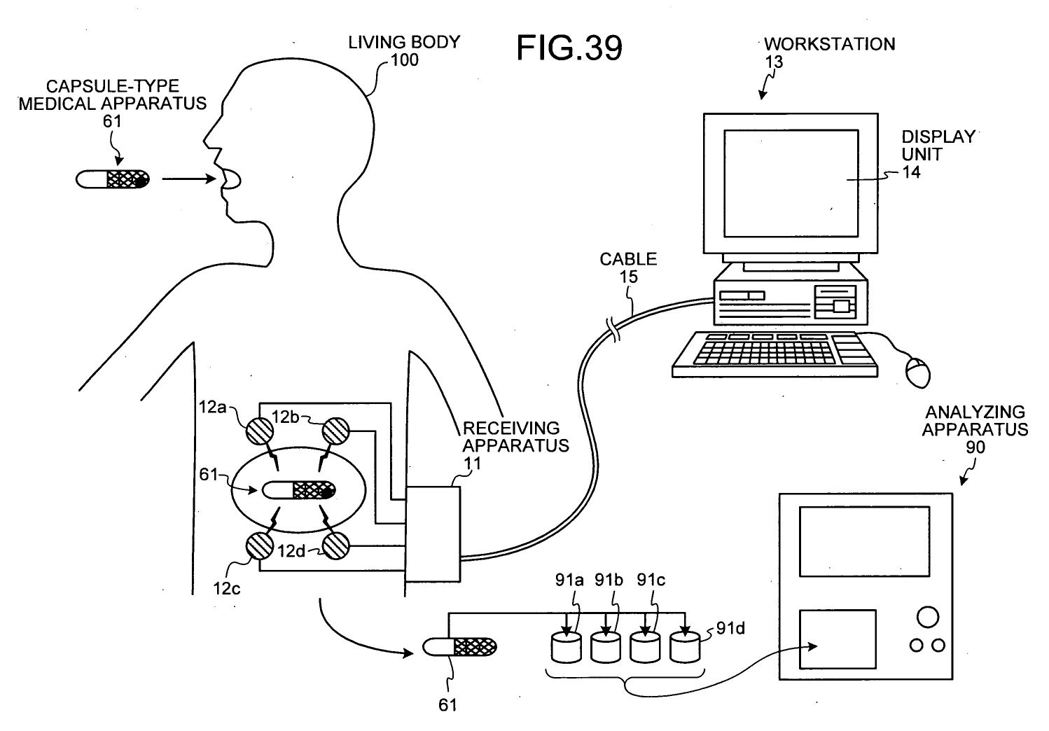

A drug delivery system including the capsule-type medical apparatus 61 according to the sixth embodiment of the present invention will be described. FIG. 39 is a schematic diagram of one exemplary configuration of the drug delivery system including the capsule-type medical apparatus 61 according to the sixth embodiment of the present invention. As shown in FIG. 39, the drug delivery system according to the sixth embodiment of the present invention has the capsule-type medical apparatus 61 in place of the capsule-type medical apparatus 51 of the drug delivery system according to the fifth embodiment. In other respects, the configuration of the drug delivery system according to the sixth embodiment is the same as that of the fifth embodiment, and the same components are denoted by the same reference characters.

In the drug delivery system according to the sixth embodiment, the capsule-type medical apparatus 61 is swallowed by the living body 100 from the mouth, and moves though the sites in the living body 100 successively or intermittently following the peristaltic movements to release the drug D1, similarly to the capsule-type medical apparatus 51 according to the fifth embodiment. At the same time, the capsule-type medical apparatus 61 in the living body 100, similarly to the capsule-type medical apparatus 51, sequentially captures the images as the drug-source information and sequentially radio transmits the captured images as the drug-source information to the receiving apparatus 11 outside.

On the other hand, the capsule-type medical apparatus 61 in the living body 100 is detained at one optional site in the living body 100, sequentially collects the body fluid at the site plural times, and stores the collected plural moieties of the body fluid separately. The capsule-type medical apparatus 61 storing the plural moieties of the body fluid is released from a detained state with respect to the site in the living body 100, and naturally excreted outside the living body 100 thereafter. The capsule-type medical apparatus 61 naturally excreted from the living body 100 is collected, and the plural moieties of the body fluid stored separately in the capsule-type medical apparatus 61 are injected into adjunctive containers 91a to 91d, respectively. Each moiety of the body fluid injected into the containers 91a to 91d is analyzed by the analyzing apparatus 90 similarly to the fifth embodiment. The analyzing apparatus 90 can output the result of successive analysis (evaluation) of an effect of the drug released to the site in the living body.

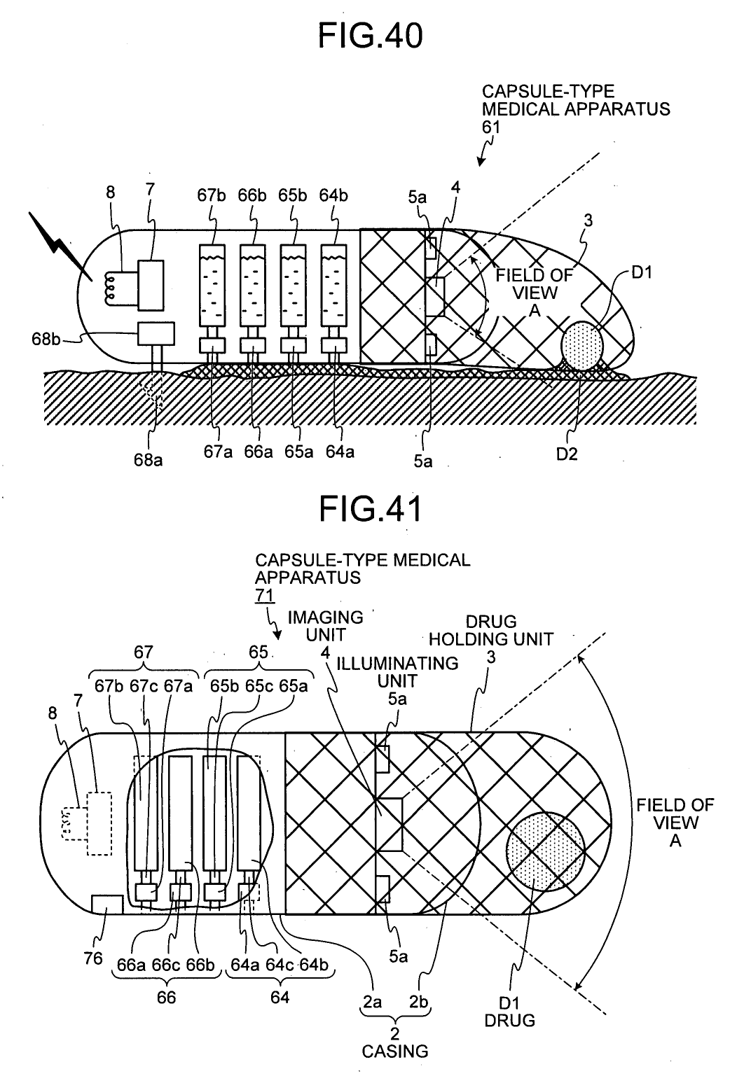

An operation of the capsule-type medical apparatus 61 collecting the body fluid at one optional site in the living body 100 plural times will be described. FIG. 40 is a schematic diagram illustrating a state of the capsule-type medical apparatus 61 according to the sixth embodiment collecting the body fluid from one site in the living body at plural times. As shown in FIG. 40, the capsule-type medical apparatus 61 inserted into the living body 100 moves through the sites in the living body 100 successively or intermittently following the peristaltic movements and the like. Thereafter, the capsule-type medical apparatus 61 is detained at one optional site in the living body 100 and collects the body fluid from the optional site plural times.

Specifically, the driving unit 68b sticks the hook 68a into the optional site in the living body 100 (e.g., a site where the drug D1 is released). The hook 68a detains the capsule-type medical apparatus 61 at one optional site in the living body 100. The pumps 64a to 67a suck (collect) the body fluid in turn every predetermined time at the site in the living body 100 where the capsule-type medical apparatus 61 is detained.

Firstly, the pump 64a sucks the body fluid at one site in the living body 100 and sends the sucked body fluid into the body-fluid storage unit 64b. After a predetermined time has passed since the start of body-fluid suction by the pump 64a, the pump 65a sucks the body fluid at the site in the living body 100 and sends the sucked body fluid into the body-fluid storage unit 65b. After a predetermined time has passed since the start of the body-fluid suction by the pump 65a, the pump 66a sucks the body fluid at the site in the living body 100 and sends the sucked body fluid into the body-fluid storage unit 66b. After a predetermined time has passed since the start of the body-fluid suction by the pump 66a, the pump 67a sucks the body fluid at the site in the living body 100 and sends the sucked body fluid into the body-fluid storage unit 67b.

The moieties of the body fluid sucked by the pumps 64a to 67a every predetermined time at plural times (plural moieties of the body fluid sequentially collected from the site where the drug D1 is released at different times) are stored in the plural body-fluid storage units 64b to 67b, respectively. The body-fluid storage units 64b to 67b hold the body fluid collected from inside the living body 100 until the body fluid is collected into the containers 91a to 91d.

As described above, the drug delivery system according to the sixth embodiment of the present invention is configured so as to have the same functions and configuration as those of the first embodiment, and so that the body fluid is sequentially collected from one optional site in the living body at plural times, the collected plural moieties of the body fluid are stored separately, and the plural moieties of the collected body fluid are analyzed separately. Therefore, in addition to the advantages and the effects of the first embodiment, the sixth embodiment has an advantage that the body fluid of the site in the living body where the drug is released can be sequentially collected at different times, and that each moiety of the body fluid collected at predetermined time intervals from the site where the drug is released can be analyzed independently. As a result, it is possible to provide a capsule-type medical apparatus and a drug delivery system including the same that have the advantages and the effects of the first embodiment, and in addition are able to acquire living-body-related medical information such as a symptom of an interior of the living body, an effect (or efficacy) of a drug, and a condition of bacteria, and particularly can successively analyze the effect of the drug released to the site in the living body.

### Seventh Embodiment

A seventh embodiment of the present invention will be described. In the sixth embodiment described above, the body fluid is collected at plural times from one optional site in the living body, and the collected plural moieties of the body fluid are stored in the plural body-fluid storage units separately. In the seventh embodiment, the body fluid is collected from each site in the living body, and the body fluid from each site is stored separately in each of the plural body-fluid collecting units.

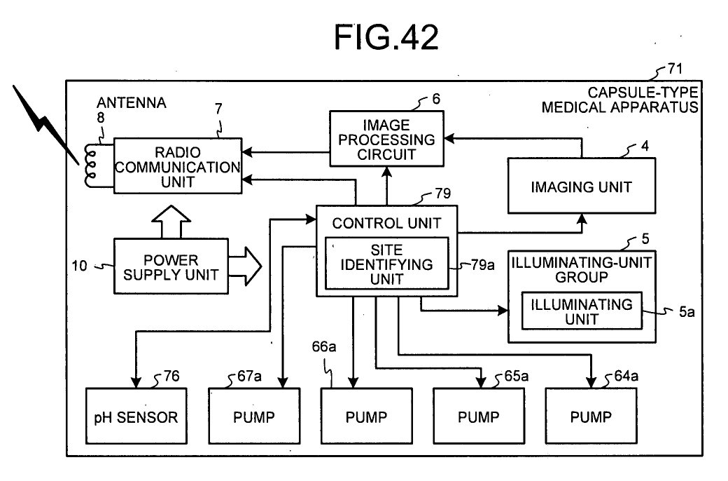

FIG. 41 is a schematic diagram of one exemplary configuration of the capsule-type medical apparatus according to the seventh embodiment of the present invention. FIG. 42 is a schematic block diagram of an exemplary configuration of the capsule-type medical apparatus according to the seventh embodiment of the present invention. As shown in FIGS. 41 and 42, the capsule-type medical apparatus 71 according to the seventh embodiment includes a pH sensor 76 and a control unit 79 in place of the detainment unit 68 and the control unit 69, respectively, of the capsule-type medical apparatus 61 according to the sixth embodiment. In other respects, the configuration of the capsule-type medical apparatus according to the seventh embodiment is the same as that of the sixth embodiment, and the same components are denoted by the same reference characters.

The pH sensor 76 sequentially detects the pH value of the body fluid to identify each site in the living body. Specifically, the pH sensor 76 is arranged near the outer wall surface of the casing main body 2a, for example, and sequentially detects the pH value of the body fluid at each site in the living body where the capsule-type medical apparatus 71 sequentially passes. The pH sensor 76 sequentially transmits the detected pH value of respective sites to the control unit 79.

The control unit 79 has a substantially similar function as that of the control unit 69 of the capsule-type medical apparatus 61 according to the sixth embodiment. The control unit 79 controls the driving of the pH sensor 76 in place of the detainment unit 76. Further, the control unit 79 sequentially controls the pumps 64a to 67a every time the site in the living body changes.

The control unit 79 has a site identifying unit 79a which identifies a current site in the living body. The site identifying unit 79a identifies the current site (such as stomach, duodenum, small intestine, and large intestine) where the capsule-type medical apparatus 71 is located based on the pH values sequentially detected by the pH sensor 76. The control unit 79 sequentially controls the pumps 64a to 67a based on a result of identification by the site identifying unit 79a.

Based on the control of the control unit 79, the pH sensor 76 sequentially detects the pH value of the body fluid which identifies the site in the living body, and sequentially transmits the detected pH value to the control unit 79. Thereafter, the pumps 64a to 67a sequentially suck (collect) the body fluid every time the site identified based on the pH value changes. The pump 64a sucks body fluid in the stomach, for example, into the body-fluid storage unit 64b; the pump 65a sucks body fluid in the duodenum, for example, into the body-fluid storage unit 65b; the pump 66a sucks body fluid in the small intestine, for example, into the body-fluid storage unit 66b; and the pump 67a sucks body fluid in the large intestine, for example, into the body-fluid storage unit 67b. Thus, the moieties of body fluid from respective sites sucked by the pumps 64a to 67a, respectively, are stored in the plural body-fluid storage units 64b to 67b, respectively.

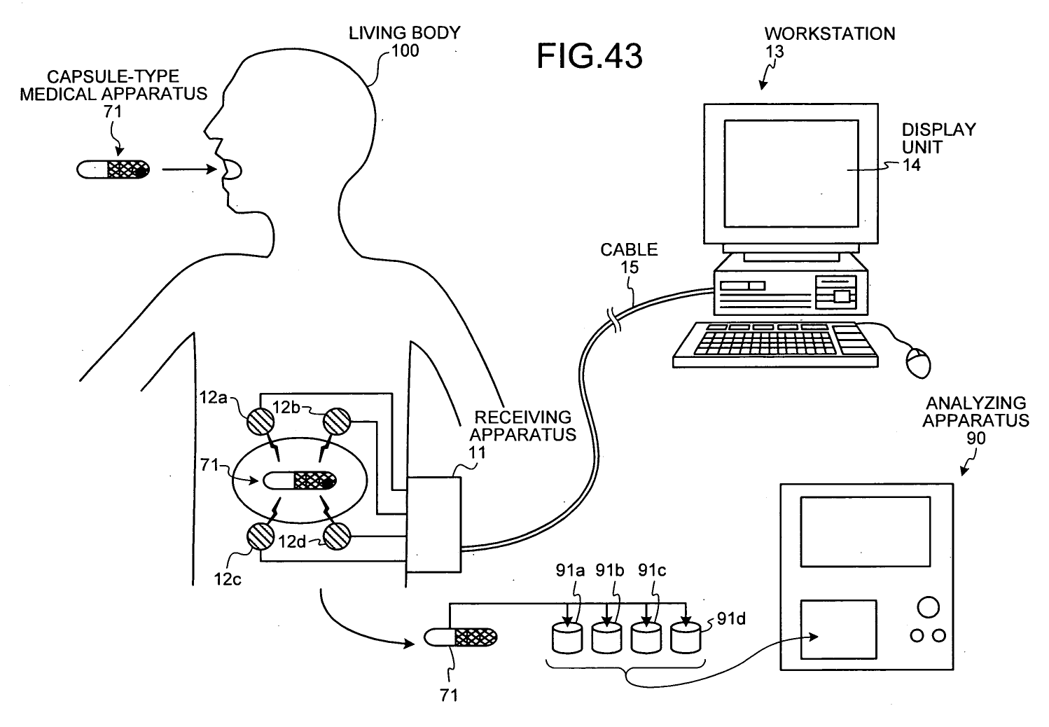

A drug delivery system including the capsule-type medical apparatus 71 according to the seventh embodiment of the present invention will be described. FIG. 43 is a schematic diagram of one exemplary configuration of the drug delivery system including the capsule-type medical apparatus 71 according to the seventh embodiment of the present invention. As shown in FIG. 43, the drug delivery system according to the seventh embodiment of the present invention includes the capsule-type medical apparatus 71 in place of the capsule-type medical apparatus 61 of the drug delivery system according to the sixth embodiment. In other respects, the configuration of the drug delivery system according to the seventh embodiment is the same as that of the sixth embodiment, and the same components are denoted by the same reference characters.

In the drug delivery system according to the seventh embodiment, the capsule-type medical apparatus 71, similarly to the capsule-type medical apparatus 61 according to the sixth embodiment, is swallowed by the living body 100 from the mouth, moves through the sites in the living body 100 successively or intermittently following the peristaltic movements and the like, to release the drug D1. At the same time, the capsule-type medical apparatus 71 in the living body 100, similarly to the capsule-type medical apparatus 61, sequentially captures the images as the drug-source information and sequentially radio transmits the captured images as the drug-source information to the receiving apparatus 11 outside.

On the other hand, the capsule-type medical apparatus 71 in the living body 100 collects the body fluid at each site in the living body 100, and stores the collected body fluid from each site separately. Thereafter, the capsule-type medical apparatus 71 storing the body fluid from each site in the living body 100 is naturally excreted to the outside of the living body 100. The capsule-type medical apparatus 71 naturally excreted from the living body 100 is collected, and the body fluid from each site separately stored in the capsule-type medical apparatus 71 is injected to corresponding one of the adjunctive containers 91a to 91d. The moieties of the body fluid from respective sites in the living body 100 injected into the containers 91a to 91d, respectively, are analyzed by the analyzing apparatus 90, respectively, similarly to the sixth embodiment. The analyzing apparatus 90 can output for each site the result of analysis (evaluation) of an effect of the drug released at each site in the living body.

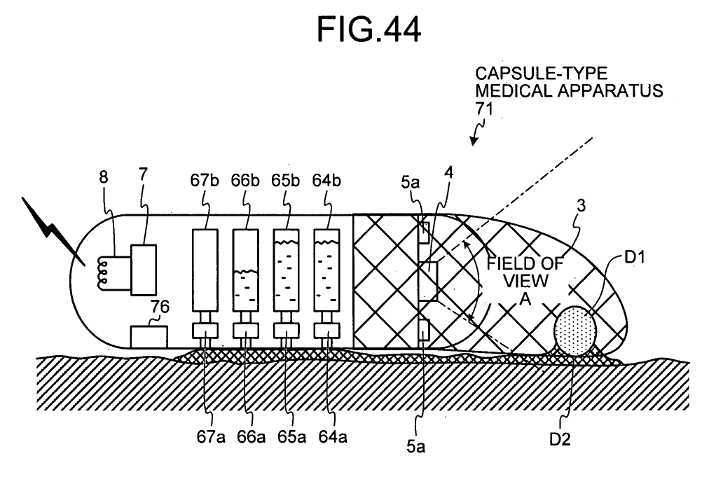

An operation of the capsule-type medical apparatus 71 collecting the body fluid from each site in the living body 100 will be described. FIG. 44 is a schematic diagram illustrating a state of the capsule-type medical apparatus 71 according to the seventh embodiment collecting the body fluid from each site in the living body. The capsule-type medical apparatus 71 inserted into the living body 100 moves through the sites in the living body 100 successively or intermittently following the peristaltic movements or the like, and collects the body fluid from each site in the living body 100 independently.

For example, as shown in FIG. 44, when the capsule-type medical apparatus 71 passes through the stomach and duodenum of the living body 100 and reaches the small intestine, the pH sensor 76 detects the pH value of the body fluid at the site (small intestine) in the living body 100, and transmits the detected pH value to the control unit 79. The site identifying unit 79a identifies the current site as a small intestine (i.e., that the capsule-type medical apparatus moves from the duodenum to the small intestine) based on the pH value detected by the pH sensor 76. The control unit 79 controls the pump 66a based on the result of determination by the site identifying unit 79a. The pump 66a sucks (collects) the body fluid from the current site (i.e., small intestine) in the living body 100 based on the control by the control unit 79. The body-fluid storage unit 66b stores the body fluid (for example, the body fluid of the small intestine) sucked by the pump 66a. At this point, the body-fluid storage unit 64b already stores the body fluid of the stomach, for example, of the living body 100, and the body-fluid storage unit 65b already stores the body fluid of the duodenum, for example, of the living body 100.

Thereafter, when the capsule-type medical apparatus 71 moves from the small intestine to the large intestine in the living body 100, the pH sensor 76, similarly to the time in the small intestine, detects the pH value of the body fluid in the large intestine of the living body 100, and the pump 67a sucks (collects) the body fluid at the current site (large intestine) in the living body 100 based on the control by the control unit 79. The body-fluid storage unit 67b stores the body fluid (e.g., body fluid of the small intestine) sucked by the pump 67a.

Thus, the body fluid of respective sites sucked at the respective sites in the living body 100 by the pumps 64a to 67a are stored in the plural body-fluid storage units 64b to 67b, respectively. The body-fluid storage units 64b to 67b hold the collected moieties of the body fluid of respective sites until the moieties of the body fluid are collected into the containers 91a to 91d.

As described above, the drug delivery system according to the seventh embodiment is configured so as to have the same function and configuration as those of the first embodiment, and to sequentially collect the body fluid at each site in the living body, store the collected body fluid of each site separately, and to analyze the collected body fluid of each site separately. Therefore, it is possible, in addition to realize the advantages and the effect of the first embodiment, to sequentially collect the body fluid at each site in the living body where the drug is released, and to analyze the body fluid of each site where the drug is released independently. As a result, it is possible to provide a capsule-type medical apparatus and a drug delivery system including the same that have the advantages and the effect of the first embodiment, and in addition, allow for acquisition of living-body-related medical information such as a symptom of the interior of the living body, an effect (or efficacy) of a drug, and a condition of bacteria, and in particular allow for an analysis (evaluation) of the effect of the drug released to each site in the living body.

In the first, fifth, sixth, and seventh embodiments described above, the drug D1 is held inside the drug holding unit 3 formed with a net-like member. The drug holding unit, however, may be formed with a porous member in which plural holes are formed so as to be able to release the drug D1 as the drug solution D2. The drug holding unit of the porous member may be provided in the casing 2, similarly to the net-like drug holding unit 3, so as to hold the drug D1 at a position within the field of view A of the imaging unit 4, and to transmit the reflected light from the site in the living body around the drug D1 to the imaging unit 4. The porous member may be such that plural holes of a suitable size to transmit the reflected light from the site in the living body to the imaging unit 4 are formed therein, or may be transparent.

Further, the drug storing unit of the porous member may block the field of view of the imaging unit 4 with respect to the site in the living body as far as the drug D1 is held in a position within the field of view A of the imaging unit 4. In this case, a site detecting unit that detects the site information indicating the site in the living body may be provided to the capsule-type medical apparatus in place of the imaging unit 4.

Further, the drug holding unit of a net-like member or a porous member may be formed detachable/attachable from/to the capsule-type medical apparatus. Then, the drug holding unit can be attached to the capsule-type medical apparatus for diagnosis such as a capsule-type endoscope which is inserted into the living body for the observation (examination) of an interior of the living body.

Further, in the third modification of the first embodiment, the drug D1 is sandwiched between two transparent holding plates. Alternatively, of the two holding plates sandwiching the drug D1, one at the casing side opposing to the imaging unit 4 may be made transparent, and the other may be made non-transparent. In this case, a site detecting unit that detects the site information indicating the site in the living body may be added to the capsule-type medical apparatus instead of the imaging unit 4.

Further, in the first embodiment, the first to the third modification thereof, and the fifth to the seventh embodiments, the imaging unit 4 captures images covering at least the drug D1 as the drug-source information indicating the release condition of the drug D1 with respect to the site in the living body. Alternatively, the detecting unit that detects the drug-source information may be, instead of the imaging unit 4, a weight-measuring unit that measures the weight of the held drug, a gap-measuring unit that measure a gap between the drug holding unit and the drug, a distance sensor that detects the dimension of the drug based on the distance to the held drug, or an ultrasonic sensor that detects the dimension and the shape of the drug by generating ultrasounds to the held drug. Further, one of a pH sensor that detects the pH value of the body fluid in the living body, a temperature-measuring unit that measures the temperature of a surrounding area of the held drug, a conductivity-measuring unit that measures the conductivity of the body fluid in the living body, and a viscosity-measuring unit that measures viscosity of the body fluid in the living body may be used. The weight-measuring unit applies vibrations to the held drug, for example, and calculates the weight of the drug based on the resonance frequency of the vibration to the drug.

Further, in the first embodiment, the first to the third modification thereof, and the second to the seventh embodiments, the imaging unit or the pH sensor is used as the site detecting unit that detects the site information indicating the site in the living body where the drug is released. Alternatively, the site detecting unit may be an imaging unit that captures an image covering at least the drug, a pH sensor that detects the pH value of the body fluid in the living body, a pressure sensor that detects pressure applied to the capsule-type medical apparatus by body tissue, a space-measuring unit that measures the width of the space surrounding the capsule-type medical apparatus, a bacteria-distribution-measuring unit that measures distribution of bacteria in the body around the capsule-type medical apparatus, and an enzyme-detecting unit that detects the enzyme present in a surrounding area of the capsule-type medical apparatus.

Alternatively, a position detecting unit may be provided in the receiving apparatus 11 outside the living body so as to detect the position of the capsule-type medical apparatus in the living body (i.e., the site in the living body where the drug is released) based on the received signal strength of the radio signals sent from the capsule-type medical apparatus and received by the plural receiving antennas distributively arranged on the body surface of the living body. In this case, the receiving apparatus 11 receives the drug-source information radio transmitted by the capsule-type medical apparatus, and the position detecting unit detects a receiving antenna which receives the drug-source information at a highest received signal strength among the plural receiving antennas, and identifies the site in the living body corresponding to the position of the detected receiving antenna as the position of the capsule-type medical apparatus. The receiving apparatus 11 transmits the drug-source information from the capsule-type medical apparatus and the site information indicating the site identified by the position detecting unit in association with each other to the workstation 13 or accumulates the same.

Further, in the first embodiment, the first to the third modification thereof, and the second to the seventh embodiments, the drug-source information and the site information radio transmitted from the capsule-type medical apparatus inserted into the living body are displayed on the display unit in real time. Alternatively, the drug-source information and the site information may not be displayed on the display unit in real time. In this case, the drug-source information and the site information radio transmitted by the capsule-type medical apparatus in the living body are sequentially accumulated in the receiving apparatus 11 outside the living body. Thereafter, the drug-source information and the site information accumulated in the receiving apparatus 11 are taken into the workstation with the use of a cable, a wireless LAN, or a portable recording medium, and the drug-source information and the site information may be displayed on the display unit 14 at a desirable time.

Alternatively, as exemplified in the fourth to the seventh embodiments, when the body fluid in the living body is collected by the body-fluid collecting unit arranged in the capsule-type medical apparatus, the body fluid from the interior of the living body may be collected from the body-fluid collecting unit of the capsule-type medical apparatus after the capsule-type medical apparatus is naturally excreted from the living body, and the collected body fluid may be analyzed, whereby the drug-source information and the site information may be acquired. In this case, the drug-source information and the site information detected through the analysis of the collected body fluid in the living body may be, for example, concentration of bacteria in the body fluid, distribution of bacteria in the body fluid, pH value of the body fluid, enzyme in the body fluid, conductivity of the body fluid, and viscosity of the body fluid. Thus, when the drug-source information and the site information are detected based on the body fluid from the living body collected from the body-fluid collecting unit, the radio communication unit may not be provided to the capsule-type medical apparatus.

Further, in the fourth embodiment, the concentration sensor 45 detects the bacteria concentration of the body fluid from the living body stored in the body-fluid storage unit 44b as the drug-source information. Alternatively, the concentration sensor 45 may detect the concentration of the drug D3 contained in the body fluid of the living body as the drug-source information.

Further, in the fourth embodiment, the drug D3 is released to the site in the living body every time the predetermined time elapses, and the pH value of the body fluid at the site in the living body is detected in synchronization with the drug release. Alternatively, the drug D3 may be sequentially released to each site in the living body. In this case, the pH sensor 46 detects the pH value of the body fluid in the living body first, and the control unit 49 identifies the current site based on the detected pH value. Every time the site in the living body as identified by the control unit 49 changes, the drug holding unit 43 releases the drug D3 to the site in the living body.

Further, in the fifth to the seventh embodiments, the capsule-type medical apparatus inserted into the living body collects the body fluid in the living body. Alternatively, the capsule-type medical apparatus may collect at least one of the body fluid, blood, and body tissue in the living body. Further, the analyzing apparatus may analyze at least one of the body fluid, blood, and body tissue in the living body as collected. In this case, it is possible to use a collecting unit which collects at least one of the body fluid, blood, and body tissue in the living body utilizing the suction force of the pump as in the body-fluid collecting unit. Alternatively, it is possible to use a collecting unit which collects at least one of the body fluid, blood, and body tissue by putting a collecting needle into a site in the living body thereby making at least one of the body fluid, blood, and body tissue in the living body adhere to the collecting needle.

Further, in the second modification of the first embodiment, the drug holding unit 17 connecting the drug D1 and the capsule-like casing 2 is exemplified as a thread-like member. Alternatively, however, the drug holding unit 17 may be a stick-like member. The drug D1 may be connected to the capsule-like casing 2 via the stick-like member. In this case, one end of the drug holding unit 17 which is a stick-like member may be adhered to the outer surface of the casing 2.

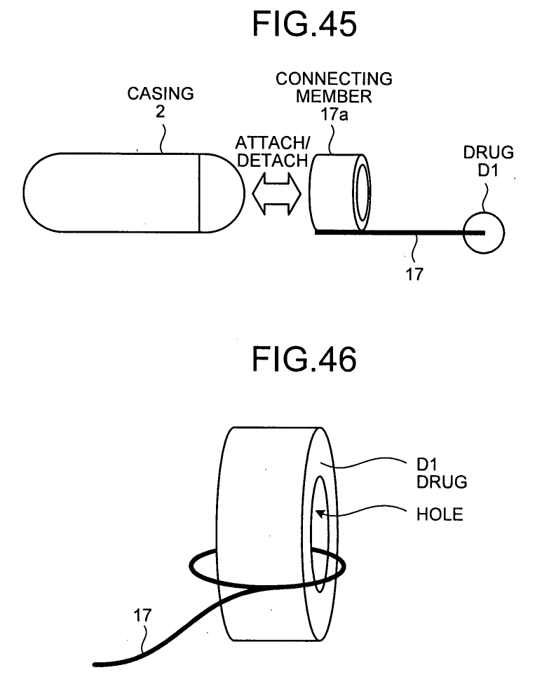

Further, in the second modification of the first embodiment, the drug D1 and the capsule-like casing 2 are connected via the drug holding unit 17. Alternatively, a ring-like or cylinder-like connecting member 17a which is detachably fit into the capsule-like casing 2 may be fixedly arranged to one end of the drug holding unit 17, as shown in FIG. 45, so that the connecting member 17a and the drug D1 are connected via the drug holding unit 17, and the connecting member 17a and the casing 2 may be fitted with each other, so that the casing 2 and the drug D1 are connected. In this case, the drug holding unit 17 may be a thread-like member, or a stick-like member.

Further, in the second modification of the first embodiment, the spherical drug D1 is held by the drug holding unit 17 in such a manner that the drug holding unit 17 penetrates the spherical drug D1. Alternatively, as shown in FIG. 46, the drug D1 may be formed to have a ring-like shape with a hole, or a cylindrical shape, and the drug holding unit 17 may be put through the hole of the drug D1 so that the drug D1 is connected to the drug holding unit 17, whereby the drug D1 may be held by the drug holding unit 17. In this case, the hole of the drug D1 may be formed substantially at the central portion of the drug D1, or may be formed off from the center of the drug D1.

The present invention has an effect that it is possible to provide a capsule-type medical apparatus and a drug delivery system including the same that can hold the drug in such a manner that the drug release such as dissolution can be achieved under the same condition as in the case where the drug is delivered to the living body by itself, that allow for confirmation of the release condition of the drug to the interior of the living body, and confirmation whether the drug is actually released to the site in the living body or not even while the drug is in the living body.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

### INDUSTRIAL APPLICABILITY

As can be seen from the foregoing, the capsule-type medical apparatus and the drug delivery system including the same according to the present invention are useful for delivery of drug to an interior of a living body such as a patient, and in particular, are suitable for a capsule-type medical apparatus and a drug delivery system including the same that allow for confirmation of whether the drug delivered to the interior of the living body is actually released to a site in the living body or not.

What is claimed is:

1. A capsule-type medical apparatus having a capsule-like casing that can be inserted into an interior of a living body and delivering a drug to a site in the living body, comprising:
a holding unit that holds the drug in such a manner that the drug can be brought into contact with a tissue in the living body; and
a detecting unit that detects a change in the drug in the living body.

2. The capsule-type medical apparatus according to claim 1, further comprising
a radio communication unit that radio transmits a result of detection of the change in the drug detected by the detecting unit to an outside.

3. The capsule-type medical apparatus according to claim 1, wherein
the detecting unit is an imaging unit that captures an image covering the drug and a tissue around the drug in the living body.

4. The capsule-type medical apparatus according to claim 1, wherein
the detecting unit includes
a light-emitting-element group in which each light-emitting element emits light of predetermined light intensity,
a light-receiving element which receives the light emitted by the light-emitting-element group,
the holding unit holds the drug between the light-emitting-element group and the light-receiving element,
the light intensity of the light emitted by the light-emitting-element group and received by the light-receiving element increases as the drug decreases, and
the light-receiving element detects the light intensity of the light which is emitted by the light emitting-element group and which increases as the drug decreases.

5. The capsule-type medical apparatus according to claim 4, wherein
the capsule-like casing includes
a light-emission-side partial casing which has the light-emitting-element group,
a light-reception-side partial casing which has the light-receiving element, and
a connecting member that connects the light-emission-side partial casing and the light-reception-side partial casing, and
the connecting member is the holding unit that holds the drug between the light-emitting-element group and the light-receiving element.

6. The capsule-type medical apparatus according to claim 1, wherein
the holding unit is a connecting member that connects the capsule-like casing and the drug.

7. The capsule-type medical apparatus according to claim 6, wherein
the connecting member is a thread-like member.

8. The capsule-type medical apparatus according to claim 6, wherein
the connecting member is a stick-like member.

9. The capsule-type medical apparatus according to claim 6, wherein
the connecting member has one end adhered to an outer surface of the capsule-like casing.

10. The capsule-type medical apparatus according to claim 6, wherein
the connecting member connects the drug by putting one end of the connecting member into a hole formed substantially at a center of the drug.

11. The capsule-type medical apparatus according to claim 6, wherein
the connecting member has one end detachably connected to the capsule-like casing.

12. The capsule-type medical apparatus according to claim 3, wherein
the holding unit includes
plural transparent plates that sandwich the drug at a predetermined position in a field of view of the imaging unit,
an elastic member that connects the plural transparent plates with each other and generates a pressing force of the plural transparent plates with respect to the drug, and
a connecting member that connects one of the plural transparent plates and the capsule-like casing.

13. The capsule-type medical apparatus according to claim 3, wherein
the holding unit includes
a drug case that houses the drug in such a manner that the drug can be brought into contact with a tissue in the living body, and
a connecting member that connects the capsule-like casing and the drug case.

14. The capsule-type medical apparatus according to claim 13, wherein
the connecting member is a shape memory member which memorizes a predetermined shape, and arranges the drug case at a position in the field of view of the imaging unit by changing to the predetermined shape under a predetermined temperature condition.

15. The capsule-type medical apparatus according to claim 1, wherein
the detecting unit is a concentration sensor that detects a drug concentration of a drug solution which is a solution of the drug in a body fluid in the living body.

16. The capsule-type medical apparatus according to claim 1, further comprising
a site detecting unit that detects site information indicating a site in the living body where the drug is changed.

17. The capsule-type medical apparatus according to claim 16, wherein
the site detecting unit is an imaging unit which captures an image covering the site in the living body.

18. The capsule-type medical apparatus according to claim 16, wherein
the site detecting unit is a pH sensor which detects pH of the interior of the living body.

19. The capsule-type medical apparatus according to claim 2, further comprising
a site detecting unit that detects site information indicating a site in the living body where the drug is changed.

20. The capsule-type medical apparatus according to claim 19, wherein
the radio communication unit radio transmits a detected change in the drug detected by the detecting unit with respect to the site of the living body and the site information detected by the site detecting unit.
